(19) United States
(12) Patent Application Publication
(10) Pub. No.: US 2026/0232818 A1
(43) Pub. Date: Aug. 13, 2026

(54) PHARMACEUTICAL COMPOSITIONS COMPRISING PROTEIN COMPLEXES

(71) Applicant: Onchilles Pharma, Inc., San Diego, CA (US)

(72) Inventors: Court R. TURNER, Rancho Santa Fe, CA (US); Lev BECKER, San Diego, CA (US); Chang CUI, Carlsbad, CA (US)

(21) Appl. No.: 19/471,684

(22) PCT Filed: Apr. 4, 2024
(86) PCT No.: PCT/US2024/022956

§ 371 Date: Oct. 1, 2025

Related U.S. Application Data
(60) Provisional application No. 63/456,916, filed on Apr. 4, 2023.
(60) Provisional application No. 63/623,030, filed on Jan. 19, 2024.

(51) Int. Cl. A61K 47/64; A61K 38/17; A61K 38/48; A61K 47/68; A61P 35/00

(52) U.S. Cl. CPC A61K 47/64; A61K 38/1722; A61K 38/482; A61K 47/6811; A61K 47/6851; A61P 35/00

(57) ABSTRACT

Provided are pharmaceutical compositions comprising alpha-2-macroglobulin (A2M) and a serine protease protein such as porcine pancreatic elastase (PPE), which are bound together in a protein complex that retains the CD95 protease cleavage and cancer-cell killing activities of the serine protease, but sterically hinders binding of the serine protease to fibrinogen and serine protease inhibitors; and related methods of use and manufacture for treating diseases such as cancers.

### CROSS-REFERENCE TO RELATED APPLICATIONS

This application claims the benefit under 35 U.S.C. § 119 (e) of U.S. Provisional Application No. 63/623,030, filed Jan. 19, 2024; and U.S. Provisional Application No. 63/456,916, filed Apr. 4, 2023, each of which is incorporated by reference in its entirety.

### STATEMENT REGARDING SEQUENCE LISTING

The Sequence Listing XML associated with this application is provided in XML file format and is hereby incorporated by reference into the specification. The name of the XML file containing the Sequence Listing XML is OPNI_009_02WO_ST26.xml. The XML file is about 28,746 bytes, was created on Mar. 28, 2024, and is being submitted electronically via USPTO Patent Center.

### BACKGROUND

### Technical Field

The present disclosure relates to pharmaceutical compositions comprising alpha-2-macroglobulin (A2M) and a serine protease protein such as porcine pancreatic elastase (PPE), which are bound together in a protein complex that retains the CD95 protease cleavage and cancer-cell killing activities of the serine protease, but sterically hinders binding of the serine protease to fibrinogen and serine protease inhibitors; and related methods of use and manufacture for treating diseases such as cancers.

### Description of the Related Art

Precision medicine, which is designed to optimize efficiency or therapeutic benefit for particular groups of patients by using genetic or molecular profiling, has gained tremendous traction for treating cancer. Identifying the specific genomic abnormalities that (i) confer risk of developing cancer, (ii) influence tumor growth, and (iii) regulate metastasis have defined how cancer is diagnosed, determined how targeted therapies are developed and implemented, and shaped cancer prevention strategies.

The need for precision medicine in cancer is largely based on the failure to identify targetable properties in tumor cells that distinguish them from healthy, non-cancer cells. Indeed, although radiation and/or chemotherapies have the capacity to effectively kill many if not most cancer cells, their efficacy is severely limited by cytotoxic effects on non-cancer cells. These findings demonstrate that rapid cell division, a property targeted by radiation therapy and chemotherapy, is not unique enough to cancer cells to achieve the specificity required to limit extensive side effects.

It has been shown that certain serine protease or elastase enzymes are selectively toxic to cancer cells but relatively non-toxic to normal or otherwise healthy cells (see, for example WO 2018/232273). However, there is a need in the art to identify optimal enzyme compositions that are capable of such selective cancer cell-toxicity but also have reduced side effects, and thereby refine the pharmacokinetics and overall clinical utility of such compositions.

### BRIEF SUMMARY

Embodiments of the present disclosure include a pharmaceutical composition, comprising a protein complex of:
(a) alpha-2-macroglobulin (A2M) proteins; and
(b) serine protease proteins,
wherein (a) and (b) are present in the composition at a molar ratio [(a):(b)] of about 1:3 to about 1:1. In certain embodiments, the A2M proteins of (a) and the serine protease proteins of (b) are bound together in the protein complex, for example, wherein the protein complex:
(i) retains CD95 (Fas Receptor) protease cleavage activity and cancer cell-killing activity of (b);
(ii) sterically hinders binding of (b) to fibrinogen and reduces or inhibits fibrinogen cleavage activity of (b); and
(iii) sterically hinders binding of (b) to serine protease inhibitors (including plasma serine protease inhibitors, for example, alpha-1 antitrypsin (A1AT)).

In some embodiments, (a) comprises, consists, or consists essentially of an amino acid sequence that is at least 80, 85, 90, 95, 98, 99, or 100% identical to a sequence selected from Table A1, or a functional fragment thereof. In some embodiments, the functional fragment thereof comprises, consists, or consists essentially of about 100, 200, 300, 400, 500, 600, 700, 800, 900, 1000, 1200, 1300, or 1400 contiguous amino acids of a sequence selected from Table A1. In some embodiments, the functional fragment thereof is composed of approximately residues 1-1400, 1-1300, 1-1200, 1-1100, 1-1000, 1-900, 1-800, 1-700, 1-600, 1-500, 1-400, 1-300, 1-200, 100-1400, 100-1300, 100-1200, 100-1100, 100-1000, 100-900, 100-800, 100-700, 100-600, 100-500, 100-400, 100-300, 100-200, 200-1400, 200-1300, 200-1200, 200-1100, 200-1000, 200-900, 200-800, 200-700, 200-600, 200-500, 200-400, 200-300, 300-1400, 300-1300, 300-1200, 300-1100, 300-1000, 300-900, 300-800, 300-700, 300-600, 300-500, 300-400, 400-1400, 400-1300, 400-1200, 400-1100, 400-1000, 400-900, 400-800, 400-700, 400-600, 400-500, 500-1400, 500-1300, 500-1200, 500-1100, 500-1000, 500-900, 500-800, 500-700, 500-600, 600-1400, 600-1300, 600-1200, 600-1100, 600-1000, 600-900, 600-800, 600-700, 700-1400, 700-1300, 700-1200, 700-1100, 700-1000, 700-900, 700-800, 800-1400, 800-1300, 800-1200, 800-1100, 800-1000, 800-900, 900-1400, 900-1300, 900-1200, 900-1100, 900-1000, 1000-1400, 1000-1300, 1000-1200, 1000-1100, 1100-1400, 1100-1300, 1100-1200, 1200-1400, or 1200-1300 of a sequence selected from Table A1.

In certain embodiments, (a) is conjugated or fused to an antibody, or an antigen binding fragment thereof. In some embodiments, the antibody, or antigen binding fragment thereof, specifically binds to a tumor-associated antigen (TAA) or tumor-specific antigen (TSA).

In some embodiments, (b) is selected from a porcine pancreatic elastase (PPE) protein, a human neutrophil elastase (ELANE) protein, a human cathepsin G (CTSG) protein, a human proteinase 3 (PR3) protein, and a granzyme B protein. In particular embodiments:
the PPE protein comprises, consists, or consists essentially of an amino acid sequence that is at least 80, 85, 90, 95, 98, 99, or 100% identical to SEQ ID NO: 5, and which retains the Q211F amino acid substitution;
the PPE protein comprises, consists, or consists essentially of an amino acid sequence that is at least 80, 85, 90, 95, 98, 99, or 100% identical to SEQ ID NO: 6, and which retains the T55A amino acid substitution;
the PPE protein comprises, consists, or consists essentially of an amino acid sequence that is at least 80, 85, 90, 95, 98, 99, or 100% identical to SEQ ID NO: 7, and which retains the Q211F and T55A amino acid substitutions;
the PPE protein comprises, consists, or consists essentially of an amino acid sequence that is at least 80, 85, 90, 95, 98, 99, or 100% identical to SEQ ID NO: 8, and which retains the N241A amino acid substitution;
the PPE protein comprises, consists, or consists essentially of an amino acid sequence that is at least 80, 85, 90, 95, 98, 99, or 100% identical to SEQ ID NO: 9, and which retains the N241Y amino acid substitution;
the PPE protein comprises, consists, or consists essentially of an amino acid sequence that is at least 80, 85, 90, 95, 98, 99, or 100% identical to SEQ ID NO: 10, and which retains the R75A amino acid substitution;
the PPE protein comprises, consists, or consists essentially of an amino acid sequence that is at least 80, 85, 90, 95, 98, 99, or 100% identical to SEQ ID NO: 11, and which retains the R75E amino acid substitution;
the PPE protein comprises, consists, or consists essentially of an amino acid sequence that is at least 80, 85, 90, 95, 98, 99, or 100% identical to SEQ ID NO: 12, and which retains the Q211A amino acid substitution;
the PPE protein comprises, consists, or consists essentially of an amino acid sequence that is at least 80, 85, 90, 95, 98, 99, or 100% identical to SEQ ID NO: 13, and which retains the R237A the PPE protein comprises, consists, or consists essentially of an amino acid sequence that is at least 80, 85, 90, 95, 98, 99, or 100% identical to SEQ ID NO: 14, and which retains the S214A amino acid substitution;
the PPE protein comprises, consists, or consists essentially of an amino acid sequence that is at least 80, 85, 90, 95, 98, 99, or 100% identical to SEQ ID NO: 15, and which retains the D74A amino acid substitution; and
the PPE protein comprises, consists, or consists essentially of an amino acid sequence that is at least 80, 85, 90, 95, 98, 99, or 100% identical to SEQ ID NO: 16.

In some embodiments:
the human ELANE protein comprises, consists, or consists essentially of an amino acid sequence that is at least 80, 85, 90, 95, 98, 99, or 100% identical to SEQ ID NO: 17;
the human CTSG protein comprises, consists, or consists essentially of an amino acid sequence that is at least 80, 85, 90, 95, 98, 99, or 100% identical to SEQ ID NO: 18;
the human PR3 protein comprises, consists, or consists essentially of an amino acid sequence that is at least 80, 85, 90, 95, 98, 99, or 100% identical to SEQ ID NO: 19; or
the human granzyme B protein comprises, consists, or consists essentially of an amino acid sequence that is at least 80, 85, 90, 95, 98, 99, or 100% identical to SEQ ID NO: 20.

In some embodiments, (a) and (b) are present in the composition at a molar ratio of about 1:3, 1:2.9, 1:2.8, 1:2.7, 1:2.6, 1:2.5, 1:2.4, 1:2.3, 1:2.1, 1:2, 1:1.9, 1:1.8, 1:1.7, 1:1.6, 1:1.5, 1:1.4, 1:1.3, 1:1.2, 1:1.1, or 1:1. In specific embodiments, (a) and (b) are present in the composition at a molar ratio of about 1:2.

Also included are methods of treating, ameliorating the symptoms of, and/or reducing the progression of, a cancer in a subject in need thereof, comprising administering to the subject a pharmaceutical composition described herein.

In some embodiments, the cancer is a primary cancer or a metastatic cancer, and is selected from one or more of melanoma (optionally metastatic melanoma), breast cancer (optionally triple-negative breast cancer, TNBC), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In some embodiments, administration (optionally intravenous administration) of the pharmaceutical composition does not substantially increase prothrombin time or partial thromboplastin time in the subject. In some embodiments, administration of the pharmaceutical composition increases cancer cell-killing in the subject by about or at least about 2-fold, 5-fold, 10-fold, 50-fold, 100-fold, 500-fold, or 1000-fold or more relative to a control or reference.

Certain embodiments include administering the pharmaceutical composition to the subject by parenteral administration. In some embodiments, the parenteral administration is intravenous administration.

Also included are methods of manufacturing a pharmaceutical composition comprising a protein complex, by combining:
(a) alpha-2-macroglobulin (A2M) proteins; and
(b) serine protease proteins,
into a composition at a molar ratio [(a):(b)] of about 1:3 to about 1:1, thereby manufacturing the pharmaceutical composition comprising the protein complex.

Certain embodiments comprise recombinantly producing (a) prior to combining with (b). Some embodiments comprise purifying (a) from plasma of a human subject prior to combining with (b). Particular embodiments comprise recombinantly producing (b) prior to combining with (a).

In some embodiments, the methods of manufacturing comprise combining (a) and (b) at a molar ratio [(a):(b)] of about 1:3, 1:2.9, 1:2.8, 1:2.7, 1:2.6, 1:2.5, 1:2.4, 1:2.3, 1:2.1, 1:2, 1:1.9, 1:1.8, 1:1.7, 1:1.6, 1:1.5, 1:1.4, 1:1.3, 1:1.2, 1:1.1, or 1:1. Specific embodiments comprise combining (a) and (b) at a molar ratio [(a):(b)] of about 1:2.

In some embodiments, the A2M proteins of (a) and the serine protease proteins of (b) are bound together in the protein complex, including wherein the protein complex:
(i) retains CD95 (Fas Receptor) protease cleavage activity and cancer cell-killing activity of (b);
(ii) sterically hinders binding of (b) to fibrinogen and reduces or inhibits fibrinogen cleavage activity of (b); and
(iii) sterically hinders binding of (b) to serine protease inhibitors (including alpha-1 antitrypsin (A1AT)).

In some embodiments. (a) comprises, consists, or consists essentially of an amino acid sequence that is at least 80, 85, 90, 95, 98, 99, or 100% identical to a sequence selected from Table A1, or a functional fragment thereof. In some embodiments, the functional fragment thereof comprises, consists, or consists essentially of about 100, 200, 300, 400, 500, 600, 700, 800, 900, 1000, 1200, 1300, or 1400 contiguous amino acids of a sequence selected from Table A1. In some embodiments, the functional fragment thereof is composed of approximately residues 1-1400, 1-1300, 1-1200, 1-1100, 1-1000, 1-900, 1-800, 1-700, 1-600, 1-500, 1-400, 1-300, 1-200, 100-1400, 100-1300, 100-1200, 100-1100, 100-1000, 100-900, 100-800, 100-700, 100-600, 100-500, 100-400, 100-300, 100-200, 200-1400, 200-1300, 200-1200, 200-1100, 200-1000, 200-900, 200-800, 200-700, 200-600, 200-500, 200-400, 200-300, 300-1400, 300-1300, 300-1200, 300-1100, 300-1000, 300-900, 300-800, 300-700, 300-600, 300-500, 300-400, 400-1400, 400-1300, 400-1200, 400-1100, 400-1000, 400-900, 400-800, 400-700, 400-600, 400-500, 500-1400, 500-1300, 500-1200, 500-1100, 500-1000, 500-900, 500-800, 500-700, 500-600, 600-1400, 600-1300, 600-1200, 600-1100, 600-1000, 600-900, 600-800, 600-700, 700-1400, 700-1300, 700-1200, 700-1100, 700-1000, 700-900, 700-800, 800-1400, 800-1300, 800-1200, 800-1100, 800-1000, 800-900, 900-1400, 900-1300, 900-1200, 900-1100, 900-1000, 1000-1400, 1000-1300, 1000-1200, 1000-1100, 1100-1400, 1100-1300, 1100-1200, 1200-1400, or 1200-1300 of a sequence selected from Table A1.

In some embodiments, (a) is conjugated or fused to an antibody, or an antigen binding fragment thereof. In some embodiments, the antibody, or antigen binding fragment thereof, specifically binds to a tumor-associated antigen (TAA) or a tumor-specific antigen (TSA).

In certain embodiments, (b) is selected from a porcine pancreatic elastase (PPE) protein, a human neutrophil elastase (ELANE) protein, a human cathepsin G (CTSG) protein, a human proteinase 3 (PR3) protein, and a human granzyme B protein.

In some embodiments:
the PPE protein comprises, consists, or consists essentially of an amino acid sequence that is at least 80, 85, 90, 95, 98, 99, or 100% identical to SEQ ID NO: 5, and which retains the Q211F amino acid substitution;
the PPE protein comprises, consists, or consists essentially of an amino acid sequence that is at least 80, 85, 90, 95, 98, 99, or 100% identical to SEQ ID NO: 6, and which retains the T55A amino acid substitution;
the PPE protein comprises, consists, or consists essentially of an amino acid sequence that is at least 80, 85, 90, 95, 98, 99, or 100% identical to SEQ ID NO: 7, and which retains the Q211F and T55A amino acid substitutions;
the PPE protein comprises, consists, or consists essentially of an amino acid sequence that is at least 80, 85, 90, 95, 98, 99, or 100% identical to SEQ ID NO: 8, and which retains the N241A amino acid substitution;
the PPE protein comprises, consists, or consists essentially of an amino acid sequence that is at least 80, 85, 90, 95, 98, 99, or 100% identical to SEQ ID NO: 9, and which retains the N241Y amino acid substitution;
the PPE protein comprises, consists, or consists essentially of an amino acid sequence that is at least 80, 85, 90, 95, 98, 99, or 100% identical to SEQ ID NO: 10, and which retains the R75A amino acid substitution;
the PPE protein comprises, consists, or consists essentially of an amino acid sequence that is at least 80, 85, 90, 95, 98, 99, or 100% identical to SEQ ID NO: 11, and which retains the R75E the PPE protein comprises, consists, or consists essentially of an amino acid sequence that is at least 80, 85, 90, 95, 98, 99, or 100% identical to SEQ ID NO: 12, and which retains the Q211A amino acid substitution;
the PPE protein comprises, consists, or consists essentially of an amino acid sequence that is at least 80, 85, 90, 95, 98, 99, or 100% identical to SEQ ID NO: 13, and which retains the R237A amino acid substitution;
the PPE protein comprises, consists, or consists essentially of an amino acid sequence that is at least 80, 85, 90, 95, 98, 99, or 100% identical to SEQ ID NO: 14, and which retains the S214A amino acid substitution;
the PPE protein comprises, consists, or consists essentially of an amino acid sequence that is at least 80, 85, 90, 95, 98, 99, or 100% identical to SEQ ID NO: 15, and which retains the D74A amino acid substitution; and
the PPE protein comprises, consists, or consists essentially of an amino acid sequence that is at least 80, 85, 90, 95, 98, 99, or 100% identical to SEQ ID NO: 16.

In some embodiments:
the human ELANE protein comprises, consists, or consists essentially of an amino acid sequence that is at least 80, 85, 90, 95, 98, 99, or 100% identical to SEQ ID NO: 17;
the human CTSG protein comprises, consists, or consists essentially of an amino acid sequence that is at least 80, 85, 90, 95, 98, 99, or 100% identical to SEQ ID NO: 18;
the human PR3 protein comprises, consists, or consists essentially of an amino acid sequence that is at least 80, 85, 90, 95, 98, 99, or 100% identical to SEQ ID NO: 19; or
the human granzyme B protein comprises, consists, or consists essentially of an amino acid sequence that is at least 80, 85, 90, 95, 98, 99, or 100% identical to SEQ ID NO: 20.

Certain methods further comprise the step or steps of testing the pharmaceutical composition in one or more activity assays selected from one or more of a CD95 cleavage assay (optionally in the presence of a serine protease inhibitor such as ALAT), a fibrinogen cleavage assay, and a cancer cell-killing assay. In some embodiments, the pharmaceutical composition cleaves CD95 (optionally in the presence of the serine protease inhibitor such as ALAT), does not substantially cleave fibrinogen, and/or has cancer cell-killing activity.

### BRIEF DESCRIPTION OF THE FIGURES

FIG. 1 illustrates the formation and structure of a protein complex comprising human A2M and a serine protease proteins. The protease binds to and cleaves the bait region of the A2M homotetramer, inducing a conformational change that incorporates the serine protease proteins into the protein complex.

FIGS. 2A-2B show the activity of Mutant F (MutF) alone in 4T1 tumor model after IV injection (day 0 and 1) on tumor growth (2A) and the number of lung metastases (2B). FIG. 2C shows the effects of MutF on prothrombin time 5 min after IV injection.

FIG. 3A shows MutF activity with different ratio of A2M:MutF in the presence of its inhibitor A1AT. FIG. 3B shows MutF activity at the 1:2 ratio of A2M:MutF in the presence of its inhibitor A1AT at different concentration. FIG. 3C shows MutF activity at the 1:2 ratio of A2M:MutF in plasma.

FIG. 4A shows MutF activity and concentration of the different fractions of A2M:MutF product after cation exchange column separation in presence or absence of ALAT. FIG. 4B shows MutF activity and concentration of the different fractions of A2M:MutF product after size exclusion column separation in presence or absence of ALAT. FIGS. 4C-4D show that A2M:MutF complexes are stable across a broad pH range as measured by enzyme activity (4C) and ALAT protection (4D).

FIG. 4E shows that A2M:MutF complexes are stable over multiple freeze/thaw cycles, as measured by enzyme activity.

FIGS. 5A-5D show MutF activity in various cells lysates after 30 min treatment in serum free media (SFM) in the presence or absence of A1AT.

FIG. 6A shows Coomassie Blue staining of CD95 cleavage after 30 min incubation with MutF or A2M:MutF at different ratios. FIG. 6B shows a Western blot for fibrinogen after 1 hr incubation with MutF or A2M:MutF at different ratios. A2M:MutF at a 1:2 ratio cleaves CD95 as efficiently as MutF alone (shown by the double band) but does not cleave fibrinogen relative to MutF alone (shown by the lower band). FIG. 6C shows the fluorescent signal of cleaved elastin following incubation with PBS, MutF alone, or A2M:MutF protein complexes.

FIG. 7A shows the prothrombin time in mouse plasma 5 min after IV injection of 480 ug of MutF or the A2M:MutF (1:2) protein complex. FIG. 7B shows the Partial Thromboplastin time in mouse plasma 5 min after IV injection in mice of 480 ug of MutF or the A2M:MutF (1:2) protein complex. FIG. 7C shows the concentration of fibrinogen in mouse plasma 5 min after IV injection of 480 ug of MutF or the A2M:MutF (1:2) protein complex.

FIG. 8A shows a cell-killing assay on various mouse cancer cell lines by MutF or the A2M:MutF (1:2) protein complex at 400 nM. FIG. 8B shows broad cytotoxicity of A2M:MutF protein complex towards cancer cells of different anatomical origin, and FIG. 8C shows that the complex does not kill non-cancer cells. FIG. 8D shows the anti-tumor effects of MutF and the A2M:MutF (1:2) protein complex in a CT26 model after 100 μg IT injection at day 0. FIG. 8E shows that A2M:MutF protein complex has an improved functional PK profile (enzymatic activity in plasma) relative to MutF alone following intravenous administration. FIG. 8F shows that A2M:MutF protein complex induces a favorable immune profile in the CT26 model (left to right in each graph is PBS, MutF, A2M:MutF). FIG. 8G shows that A2M:MutF protein complex elicits a tumor antigen-specific CD8+ T cell response in the CT26 model (left to right in each graph is PBS, MutF, A2M:MutF).

FIG. 9A shows that A2M:MutF protein complex, in contrast to doxorubicin and oxaliplatin, has a wide therapeutic window as shown by killing human ovarian cancer cells without killing non-cancer cells from patients. FIG. 9B shows that A2M:MutF comparably kills cancer cells isolated from chemo-naïve and chemo-treated patients, in contrast to doxorubicin and oxaliplatin, which show reduced killing of cancer cells isolated from chemo-treated relative to chemo-naïve patients.

FIG. 10A shows that A2M:MutF protein complex induces ICD markers in CT26 and A549 cells. FIG. 10B shows that A2M:MutF protein complex induces ICD markers in human ovarian patient-derived tumor cells (left to right in each graph are CTRL, A2M:MutF, oxaliplatin).

FIGS. 11A-11B show tumor growth post-treatment in the mouse CT26 tumor model. FIG. 11C shows tumor weight at 15 days post-treatment (11C from left to right: vehicle every other day, A2M:MutF 100 μg daily, A2M:MutF 200 μg every other day, A2M:MutF 400 μg every 4th day).

FIGS. 12A-12B show that A2M:MutF protein complex effectively attenuates tumor growth in Jh-BALB/c CT26 colorectal cancer model. FIGS. 12C-12D show that A2M:MutF protein complex treats primary tumor and metastasis in a Jh-C57BL/6 B16F10 melanoma model. FIG. 12E shows that A2M:MutF protein complex displays efficacy across a range of tumors with variable immunological status.

FIGS. 13A-13C show that A2M:MutF protein complex has improved anti-tumor efficacy relative to SoC chemotherapy (oxaliplatin) without observed toxicity.

FIG. 14A shows the efficacy of A2M:MutF protein complex in a lung cancer human xenograft model, FIG. 14B summarizes the efficacy of A2M:MutF protein complex across a variety of prostate cancer, colon cancer, and lung cancer models. FIG. 14C shows that A2M:MutF protein complex effectively kills human ovarian patient-derived tumor cells (from patient CDX_002) in a xenograft mouse model, and FIG. 14D summarizes the efficacy of A2M:MutF protein complex in this model across three ovarian cancer patients. FIGS. 14E-14F show that A2M:MutF protein complex effectively kills patient-derived breast cancer cells in vitro and in vivo. FIG. 14G summarizes the in vivo efficacy of A2M:MutF protein complex across a variety of human tumors and shows that the efficacy is independent of tumor genetics or immune status.

FIG. 15 shows that mice treated with A2M:MutF protein complexes are tumor-free (5/11) after initial challenge with CT26 colorectal cancer cells, and that all of these mice (5/5) retain their tumor-free status upon re-challenge with CD26 cells.

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

An “antagonist” refers to a biological or chemical agent that interferes with or otherwise reduces the physiological action of another agent or molecule. In some instances, the antagonist specifically binds to the other agent or molecule. Included are full and partial antagonists.

An “agonist” refers to a biological or chemical agent that increases or enhances the physiological action of another agent or molecule. In some instances, the agonist specifically binds to the other agent or molecule. Included are full and partial agonists.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “half maximal effective concentration” or “EC₅₀” refers to the concentration of an agent (for example, a protein complex) as described herein at which it induces a response halfway between the baseline and maximum after some specified exposure time; the EC₅₀ of a graded dose response curve therefore represents the concentration of a compound at which 50% of its maximal effect is observed. EC₅₀ also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC50” and the Hill slope, or it can be determined from the data directly, using routine knowledge in the art. In some embodiments, the EC₅₀ of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

The “half-life” of an agent can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The term “heterologous” refers to a feature or element in a polypeptide or encoding polynucleotide that is derived from a different source than the wild-type polypeptide or encoding polynucleotide, for example, a feature from a different species than the wild-type, or a non-natural, engineered feature.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is about or at least about 1.1, 1.2, 1.5, 2, 3, 4, 5, 6, 7, 8, 9, 10, 15, 20, 30, 40, 50, 60, 70, 80, 90, 100, 200, 300, 400, 500, or 1000-fold more than the amount produced by no composition (e.g., the absence of agent) or a control composition. A “decreased” or “reduced” amount is typically a “statistically significant” amount, and may include a decrease that about or at least about 1.1, 1.2, 1.5, 2, 3, 4, 5, 6, 7, 8, 9, 10, 15, 20, 30, 40, 50, 60, 70, 80, 90, 100, 200, 300, 400, 500, or 1000-fold less than the amount produced by no composition (e.g., the absence of an agent) or a control composition. Examples of comparisons and “statistically significant” amounts are described herein.

The terms “polypeptide,” “protein”, and “peptide” are used interchangeably and refer to a polymer of amino acids not limited to any particular length. The term “enzyme” includes polypeptide or protein catalysts. As used herein a “proprotein”, “proenzyme”, or “zymogen” refers to an inactive (or substantially inactive) protein or enzyme, which typically is activated by protease cleavage of an activation peptide to generate an active protein or enzyme. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, RNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotide, regardless of the length of the coding sequence itself, may be combined with other sequences, for example, expression control sequences.

“Expression control sequences” include regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, which have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5) direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Eukaryotic promoters can often, but not always, contain “TATA” boxes and “CAT” boxes. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types), and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include the Tet system, (U.S. Pat. Nos. 5,464,758 and 5,814,618), the Ecdysone inducible system (No et al., Proc. Natl. Acad. Sci. (1996) 93 (8): 3346-3351; the T-REX™ system (Invitrogen Carlsbad, CA), LacSwitch® (Stratagene, (San Diego, CA) and the Cre-ERT tamoxifen inducible recombinase system (Indra et al. Nuc. Acid. Res. (1999) 27 (22): 4324-4327; Nuc. Acid. Res. (2000) 28 (23): e99; U.S. Pat. No. 7,112,715; and Kramer & Fussenegger, Methods Mol. Biol. (2005) 308:123-144) or any promoter known in the art suitable for expression in the desired cells.

An “expressible polynucleotide” includes a cDNA, RNA, mRNA or other polynucleotide that comprises at least one coding sequence and optionally at least one expression control sequence, for example, a transcriptional and/or translational regulatory element, and which can express an encoded polypeptide upon introduction into a cell.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the proteins/polypeptides described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and which substantially retain at least one activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent described herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise,

### Protein Complexes

Embodiments of the present disclosure relate generally to pharmaceutical compositions, comprising protein complexes of alpha-2-macroglobulin (A2M) proteins and serine protease proteins such as PPE. Here, certain serine proteases are able to kill cancer cells upon direct contact with or administration to tumors (e.g., intra-tumoral administration), irrespective of their genetic abnormalities, and are relatively harmless to non-cancerous or healthy cells (see, for example, WO 2018/232273; WO/2020/132465; PCT/US2021/046453; and PCT/2021/046467). However, in some instances, the systemic administration of standalone serine proteases such as PPE can negatively impact coagulation, for example, by inducing cleavage of fibrinogen. The present disclosure relates in part to the discovery that protein complexes of human A2M and serine proteases such as PPE not only sterically protect the serine protease from serine protease inhibitors in plasma while retaining CD95 protease cleavage and cancer cell-killing activity, but also reduce the negative impact of the serine protease on coagulation, for instance, by sterically inhibiting or otherwise reducing the ability of the complexed serine protease to cleave fibrinogen. The present disclosure further relates to the discovery of a range of optimal molar ratios between [A2M proteins]: [serine protease proteins] in the protein complex that provides a balance between retaining the CD95 cleavage and cancer cell-killing activity of the serine protease and reducing its negative impact on coagulation, as measured, for example, by reduced prothrombin time or decreased cleavage of fibrinogen relative to the serine protease alone.

Certain embodiments thus include pharmaceutical compositions, comprising a protein complex of: (a) alpha-2-macroglobulin (A2M) proteins; and (b) serine protease proteins, wherein (a) and (b) are present in the composition at a molar ratio [(a):(b)] of about 1:3 to about 1:1, including wherein the A2M proteins of (a) and the serine protease proteins of (b) are bound together in the protein complex. In some embodiments, the protein complex: (i) retains the CD95 (Fas Receptor) protease cleavage activity and cancer cell-killing activity of (b); (ii) sterically hinders binding of (b) to fibrinogen, thereby reducing or inhibiting the fibrinogen cleavage activity of (b); and (iii) sterically hinders binding of (b) to serine protease inhibitors such as alpha-1 antitrypsin (A1AT), thereby protecting (b) from inhibition by the serine protease inhibitors.

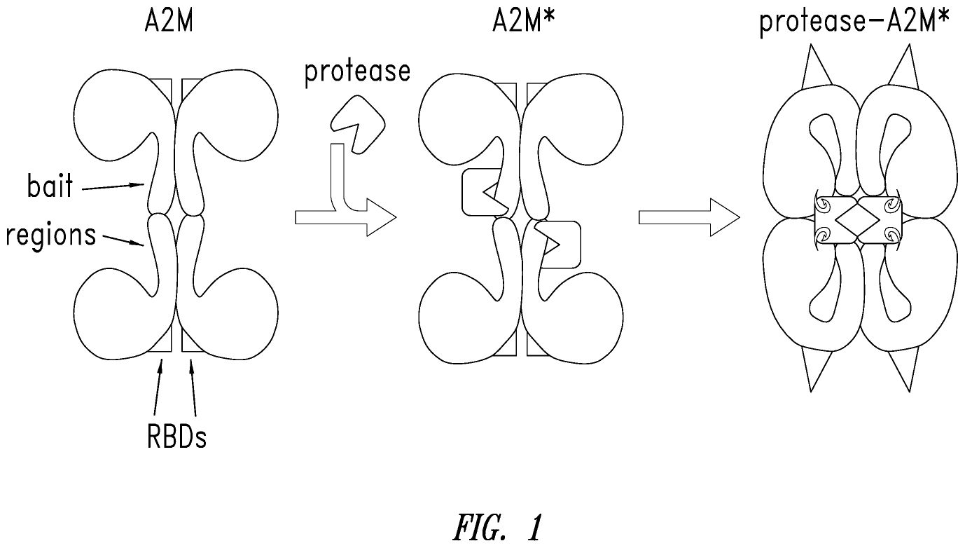

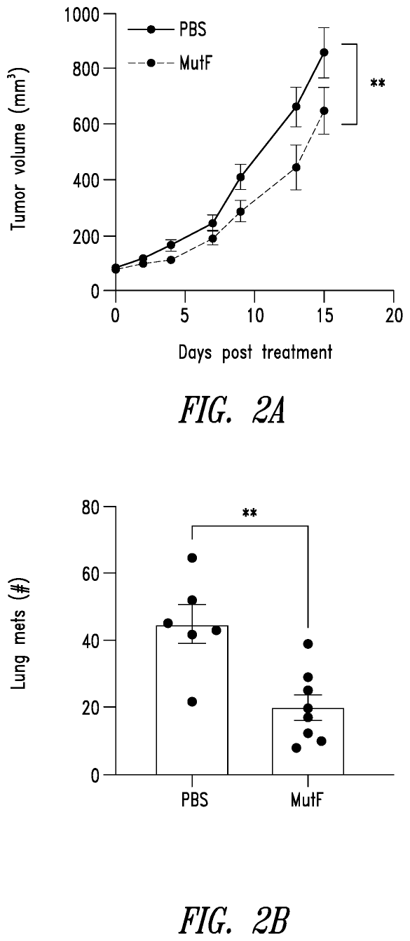

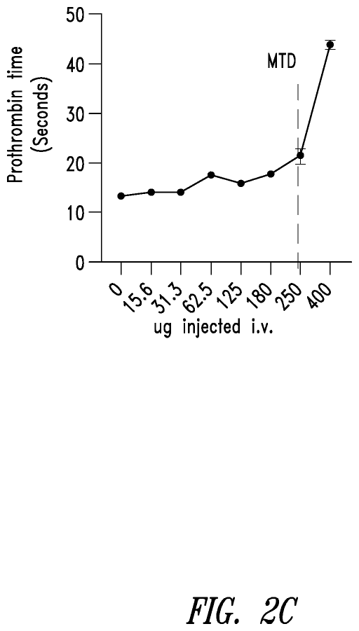

In certain embodiments, the A2M proteins are bound together in the protein complex as A2M monomers or A2M multimers, for example, as A2M dimers such as A2M homodimers, as A2M trimers such as A2M homotrimers, or as A2M tetramers such as A2M homotetramers. In specific embodiments, the A2M proteins of (a) are bound together as A2M homotetramers, and the serine protease proteins of (b) are bound by the A2M homotetramers into the protein complex. In specific embodiments, each protein complex is composed of one set of A2M homotetramers (that is, four A2M proteins, either as (i) four whole A2M proteins or as (ii) up to eight A2M fragments which result from cleavage of the bait region of the whole A2M proteins by the serine protease(s) as the latter join the A2M homotetramer complex) and two serine protease proteins (see, for example, FIG. 1).

In certain embodiments, the protein complex sterically hinders binding of (b) to larger molecules but does not sterically hinder binding of (b) to smaller molecules. That is, as noted above, the protein complex sterically hinders binding of (b) to larger molecules such as serine protease proteins (e.g., A1AT), fibrinogen, and/or plasma antibodies. Thus, in some embodiments, the protein complex protects (b) from inhibition by serine proteases and also reduces/inhibits the ability of (b) to cleave fibrinogen. In some instances, for example, where the serine protease of (b) is a non-human protein such as PPE, the protein complex protects (b) from anti-PPE plasma antibodies or the generation of anti-PPE plasma antibodies. In some instances, such provide clinical advantages in the administration of non-human protein drugs such as PPE to humans, which can otherwise generate anti-drug antibodies. In contrast, the protein complex does not sterically hinder binding of (b) to smaller molecules such as CD95. Thus, in specific embodiments, (b) in the protein complex cleaves CD95 and kills cancer cells but does not substantially cleave fibrinogen.

The pharmaceutical composition and protein complexes described herein comprise an alpha-2-macroglobulin (A2M) protein, for example, a human A2M protein. A2M is a highly conserved protease inhibitor present in plasma at relatively high concentrations (0.1-6 mg/ml) (Bhattacharjee et al., J. Biol. Chem. 275:26806-11, 2000). It often exists as a tetramer of four identical ~180 kDa subunits that forms a hollow cylinder-like structure. It can present multiple target peptide bonds to attacking proteases in its central “bait” domain. Human A2M “traps” serine proteases such as PPE: here, after the serine protease binds to and cleaves the bait region, a conformational change is induced in A2M which traps the serine protease in a way that the protease remains active against low molecular weight substrates but has significantly reduced activity against high molecular weight substrates (see, for example, Vandooren and Itoh, Frontiers in Immunology, 12, 2021; and Harwood et al., Molecular & Cellular Proteomics, 20, 2021). The amino acid sequence of full-length and mature (w/o signal peptide) human A2M is provided in Table A1 below.

TABLE A1
A2M Proteins

| | | SEQ ID |
|---|---|---|
| Protein | Sequence | NO : |
| Human A2M | MGKNKLLHPSLVLLLLVLLPTDASVSGKPQYMVLVPSLLHTETTEKGCVLLSYL | 22 |
| Full-Length | NETVTVSASLESVRGNRSLFTDLEAENDVLHCVAFAVPKSSSNEEVMFLTVQVK | |
| signal | GPTQEFKKRTTVMVKNEDSLVFVQTDKSIYKPGQTVKFRVVSMDENFHPLNELI | |
| peptide | PLVYIQDPKGNRIAQWQSFQLEGGLKQFSFPLSSEPFQGSYKVVVQKKSGGRTE | |
| underlined | HPFTVEEFVLPKFEVQVTVPKIITILEEEMNVSVCGLYTYGKPVPGHVTVSICR | |
| bait region | KYSDASDCHGEDSQAFCEKFSGQLNSHGCFYQQVKTKVFQLKRKEYEMKLHTEA | |
| bold and | QIQEEGTVVELTGRQSSEITRTITKLSFVKVDSHFRQGIPFFGQVRLVDGKGVP | |
| underlined | IPNKVIFIRGNEANYYSNATTDEHGLVQFSINTTNVMGTSLTVRVNYKDRSPCY | |
| | GYQWVSEEHEEAHHTAYLVFSPSKSFVHLEPMSHELPCGHTQTVQAHYILNGGT | |
| | LLGLKKLSFYYLIMAKGGIVRTGTHGLLVKQEDMKGHFSISIPVKSDIAPVARL | |
| | LIYAVLPTGDVIGDSAKYDVENCLANKVDLSFSPSQSLPASHAHLRVTAAPQSV | |
| | CALRAVDQSVLLMKPDAELSASSVYNLLPEKDLTGFPGPLNDQDNEDCINRHNV | |
| | YINGITYTPVSSTNEKDMYSFLEDMGLKAFTNSKIRKPKMCPQLQQYEMKGPEG | |
| | LRVGFYESDVMGRGHARLVHVEEPHTETVRKYFPETWIWDLVVVNSAGVAEVGV | |
| | TVPDTITEWKAGAFCLSEDAGLGISSTASERAFQPFFVELTMPYSVIRGEAFTL | |
| | KATVINYLPKCIRVSVQLEASPAFLAVPVEKEQAPHCICANGRQTVSWAVTPKS | |
| | LGNVNFTVSAEALESQELCGTEVPSVPEHGRKDTVIKPLLVEPEGLEKETTENS | |
| | LLCPSGGEVSEELSLKLPPNVVEESARASVSVLGDILGSAMQNTQNLLQMPYGC | |
| | GEQNMVLFAPNIYVLDYLNETQQLTPEIKSKAIGYLNTGYQRQLNYKHYDGSYS | |
| | TFGERYGRNQGNTWLTAFVLKTFAQARAYIFIDEAHITQALIWLSQRQKDNGCF | |
| | RSSGSLLNNAIKGGVEDEVTLSAYITIALLEIPLTVTHPVVRNALFCLESAWKT | |
| | AQEGDHGSHVYTKALLAYAFALAGNQDKRKEVLKSLNEEAVKKDNSVHWERPQK | |
| | PKAPVGHFYEPQAPSAEVEMTSYVLLAYLTAQPAPTSEDLTSATNIVKWITKQQ | |
| | NAQGGFSSTQDTVVALHALSKYGAATFTRTGKAAQVTIQSSGTFSSKFQVDNNN | |
| | RLLLQQVSLPELPGEYSMKVTGEGCVYLQTSLKYNILPEKEEFPFALGVQTLPQ | |
| | TCDEPKAHTSFQISLSVSYTGSRSASNMAIVDVKMVSGFIPLKPTVKMLERSNH | |
| | VSRTEVSSNHVLIYLDKVSNQTLSLFFTVLQDVPVRDLKPAIVKVYDYYETDEF | |
| | AIAEYNAPCSKDLGNA | |
| Human A2M | SVSGKPQYMVLVPSLLHTETTEKGCVLLSYLNETVTVSASLESVRGNRSLFTDL | 23 |
| Mature | EAENDVLHCVAFAVPKSSSNEEVMFLTVQVKGPTQEFKKRTTVMVKNEDSLVFV | |
| bait region | QTDKSIYKPGQTVKFRVVSMDENFHPLNELIPLVYIQDPKGNRIAQWQSFQLEG | |
| bold and | GLKQFSFPLSSEPFQGSYKVVVQKKSGGRTEHPFTVEEFVLPKFEVQVTVPKII | |
| underlined | TILEEEMNVSVCGLYTYGKPVPGHVTVSICRKYSDASDCHGEDSQAFCEKESGQ | |
| | LNSHGCFYQQVKTKVFQLKRKEYEMKLHTEAQIQEEGTVVELTGRQSSEITRTI | |
| | TKLSFVKVDSHFRQGIPFFGQVRLVDGKGVPIPNKVIFIRGNEANYYSNATTDE | |
| | HGLVQFSINTTNVMGTSLTVRVNYKDRSPCYGYQWVSEEHEEAHHTAYLVFSPS | |
| | KSFVHLEPMSHELPCGHTQTVQAHYILNGGTLLGLKKLSFYYLIMAKGGIVRTG | |
| | THGLLVKQEDMKGHFSISIPVKSDIAPVARLLIYAVLPTGDVIGDSAKYDVENC | |
| | LANKVDLSFSPSQSLPASHAHLRVTAAPQSVCALRAVDQSVLLMKPDAELSASS | |
| | VYNLLPEKDLTGFPGPLNDQDNEDCINRHNVYINGITYTPVSSTNEKDMYSFLE | |
| | DMGLKAFTNSKIRKPKMCPQLQQYEMHGPEGLRVGFYESDVMGRGHARLVHVEE | |
| | PHTETVRKYFPETWIWDLVVVNSAGVAEVGVTVPDTITEWKAGAFCLSEDAGLG | |
| | ISSTASLRAFQPFFVELTMPYSVIRGEAFTLKATVLNYLPKCIRVSVQLEASPA | |
| | FLAVPVEKEQAPHCICANGRQTVSWAVTPKSLGNVNFTVSAEALESQELCGTEV | |
| | PSVPEHGRKDTVIKPLLVEPEGLEKETTFNSLLCPSGGEVSEELSLKLPPNVVE | |
| | ESARASVSVLGDILGSAMQNTQNLLQMPYGCGEQNMVLFAPNIYVLDYLNETQQ | |
| | LTPEIKSKAIGYLNTGYQRQLNYKHYDGSYSTFGERYGRNQGNTWLTAFVLKTF | |
| | AQARAYIFIDEAHITQALIWLSQRQKDNGCFRSSGSLLNNAIKGGVEDEVTLSA | |
| | YITIALLEIPLTVTHPVVRNALFCLESAWKTAQEGDHGSHVYTKALLAYAFALA | |
| | GNQDKRKEVLKSLNEEAVKKDNSVHWERPQKPKAPVGHFYEPQAPSAEVEMTSY | |
| | VLLAYLTAQPAPTSEDLTSATNIVKWITKQQNAQGGFSSTQDTVVALHALSKYG | |
| | AATFTRTGKAAQVTIQSSGTFSSKFQVDNNNRELLQQVSLPELPGEYSMKVTGE | |
| | GCVYLQTSLKYNILPEKEEFPFALGVQTLPQTCDEPKAHTSFQISLSVSYTGSR | |
| | SASNMAIVDVKMVSGFIPLKPTVKMLERSNHVSRTEVSSNHVLIYLDKVSNQTL | |
| | SLFFTVLQDVPVRDLKPAIVKVYDYYETDEFAIAEYNAPCSKDLGNA | |

Thus, in some embodiments, the A2M protein portion of the protein complex comprises, consists, or consists essentially of an amino acid sequence that is at least 80, 85, 90, 95, 98, 99, or 100% identical to a sequence selected from Table A1, or a functional fragment thereof. In some embodiments, the functional fragment thereof comprises, consists, or consists essentially of about 100, 200, 300, 400, 500, 600, 700, 800, 900, 1000, 1200, 1300, or 1400 contiguous amino acids of a sequence selected from Table A1.

For instance, in specific embodiments, the functional fragment thereof is composed of approximately residues 1-1400, 1-1300, 1-1200, 1-1100, 1-1000, 1-900, 1-800, 1-700, 1-600, 1-500, 1-400, 1-300, 1-200, 100-1400, 100-1300, 100-1200, 100-1100, 100-1000, 100-900, 100-800, 100-700, 100-600, 100-500, 100-400, 100-300, 100-200, 200-1400, 200-1300, 200-1200, 200-1100, 200-1000, 200-900, 200-800, 200-700, 200-600, 200-500, 200-400, 200-300, 300-1400, 300-1300, 300-1200, 300-1100, 300-1000, 300-900, 300-800, 300-700, 300-600, 300-500, 300-400, 400-1400, 400-1300, 400-1200, 400-1100, 400-1000, 400-900, 400-800, 400-700, 400-600, 400-500, 500-1400, 500-1300, 500-1200, 500-1100, 500-1000, 500-900, 500-800, 500-700, 500-600, 600-1400, 600-1300, 600-1200, 600-1100, 600-1000, 600-900, 600-800, 600-700, 700-1400, 700-1300, 700-1200, 700-1100, 700-1000, 700-900, 700-800, 800-1400, 800-1300, 800-1200, 800-1100, 800-1000, 800-900, 900-1400, 900-1300, 900-1200, 900-1100, 900-1000, 1000-1400, 1000-1300, 1000-1200, 1000-1100, 1100-1400, 1100-1300, 1100-1200, 1200-1400, or 1200-1300 of a sequence selected from Table A1. In some embodiments, the functional fragment thereof is capable of forming an A2M homotetramer and trapping or otherwise binding the serine protease (such as PPE) into the protein complex in a configuration which sterically hinders binding of the serine protease to serine protease inhibitors such as ALAT, retains CD95 protease cleavage activity and cancer-cell killing activity of the serine protease, and/or inhibits or otherwise reduces the ability of the serine protease to cleave fibrinogen.

In certain embodiments, the A2M portion of the protein complex improves uptake into cancer cells relative to the serine protease alone. For instance, A2M binds to the LPR1 and GRP78 receptors, which are expressed on normal and cancer cells. Indeed, elevated GRP78 levels generally correlate with higher pathologic grade, recurrence, and poor patient survival in breast, liver, prostate, colon, and gastric cancers (see, for example, Lee, Cancer Res. 67:3496-3499, 2007), and in some instances the ability of A2M to bind GRP78 improves selective targeting to cancer cells that express GRP78.

In some embodiments, the A2M portion of the protein complex is fused or otherwise conjugated to an antibody, or an antigen binding fragment thereof. In some embodiments, the antibody, or antigen binding fragment thereof, specifically binds to a tumor-associated antigen (TAA) or tumor-specific antigen (TSA). Exemplary TAAs and TSAs include, without limitation, alphafetoprotein (AFP), epithelial tumor antigen (ETA), tyrosinase, human Her2/neu, Her1/EGF receptor (EGFR), Her3, A33 antigen, B7H3, CD5, CD19, CD20, CD22, CD23 (IgE Receptor), melanoma associated antigen (MAGE), C242 antigen, 5T4, IL-6, IL-13, vascular endothelial growth factor VEGF (e.g., VEGF-A) VEGFR-1, VEGFR-2, VEGR-3, NRP2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-IR), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PSMA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

The pharmaceutical composition and protein complexes described herein comprise a serine protease protein. Examples of serine proteases include porcine pancreatic elastase (PPE), human neutrophil elastase (ELANE), human cathepsin G (CTSG), human proteinase 3 (PR3), and human granzyme B (GZMB). The amino acid sequences of exemplary full-length, wild-type serine protease proproteins are provided in Table S1 below.

TABLE S1
Full-Length Serine Protease Sequences

| | | SEQ ID |
|---|---|---|
| Name | Sequence | NO: |
| WT PPE | MLRLLVVASLVLYGHSTQDFPETNARVVGGTEAQRNSWPSQISLQY | 1 |
| proprotein, | RSGSSWAHTCGGTLIRQNWVMTAAHCVDRELTFRVVVGEHNLNQND | |
| signal peptide | GTEQYVGVQKIVVHPYWNTDDVAAGYDIALLRLAQSVTLNSYVQLG | |
| underlined | VLPRAGTILANNSPCYITGWGLTRINGQLAQTLQQAYLPTVDYAIC | |
| | SSSSYWGSTVKNSMVCAGGDGVRSGCQGDSGGPLHCLVNGQYAVHG | |
| | VTSFVSRLGCNVTRKPTVFTRVSAYISWINNVIASN | |
| WT human ELANE, | MTLGRRLACLFLACVLPALLLGGTALASEIVGGRRARPHAWPEMVS | 2 |
| peptidase domain | LQLRGGHFCGATLIAPNFVMSAAHCVANVNVRAVRVVLGAHNLSRR | |
| residues 30-247, | EPTRQVFAVQRIFENGYDPVNLLNDIVILQLNGSATINANVQVAQL | |
| or residues 30- | PAQGRRLGNGVQCLAMGWGLLGRNRGIASVLQELNVTVVTSLCRRS | |
| 267, | NVCTLVRGRQAGVCFGDSGSPLVQNGLIHGIASFVRGGCASGLYPD | |
| signal peptide | AFAPVAQFVNWIDSIIQRSEDNPCPHPRDPDPASRTH | |
| underlined | | |
| WT human CTSG, | MQPLLLLLAFLLPTGAEAGEIIGGRESRPHSRPYMAYLQIQSPAGQ | 3 |
| peptidase domain | SRCGGFLVREDFVLTAAHCWGSNINVTLGAHNIQRRENTQQHITAR | |
| residues 21-243, | RAIRHPQYNQRTIQNDIMLLQLSRRVRRNRNVNPVALPRAQEGLRP | |
| or residues 21- | GTLCTVAGWGRVSMRRGTDTLREVQLRVQRDRQCLRIFGSYDPRRQ | |
| 255, | ICVGDRRERKAAFKGDSGGPLLCNNVAHGIVSYGKSSGVPPEVFTR | |
| signal peptide | VSSFLPWIRTTMRSFKLLDQMETPL | |
| underlined | | |
| WT Human PR3, | MAHRPPSPALASVLLALLLSGAARAAEIVGGHEAQPHSRPYMASLQ | 4 |
| peptidase domain | MRGNPGSHFCGGTLIHPSFVLTAAHCLRDIPQRLVNVVLGAHNVRT | |
| residues 28-248, | QEPTQQHFSVAQVFLNNYDAENKINDVLLIQLSSPANLSASVATVQ | |
| signal peptide | LPQQDQPVPHGTQCLAMGWGRVGAHDPPAQVLQELNVTVVTFFCRP | |
| underlined | HNICTFVPRRKAGICFGDSGGPLICDGIIQGIDSFVIWGCATRLFP | |
| | DFFTRVALYVDWIRSTLRRVEAKGRP | |
| WT Human | MQPILLLLAFLLLPRADAGELIGGHEAKPHSRPYMAYLMIWDQKSL | 21 |
| granzyme B, | KRCGGFLIRDDFVLTAAHCWGSSINVTLGAHNIKEQEPTQQFIPVK | |
| peptidase domain | RPIPHPAYNPKNFSNDIMLLQLERKAKRTRAVQPLRLPSNKAQVKP | |
| residues 21-245, | GQTCSVAGWGQTAPLGKHSHTLQEVKMTVQEDRKCESDLRHYYDST | |
| signal peptide | IELCVGDPEIKKTSFKGDSGGPLVCNKVAQGIVSYGRNNGMPPRAC | |
| underlined | TKVSSFVHWIKKTMKRY | |

Thus, in certain embodiments, the serine protease protein comprises, consists, or consists essentially of a full-length, serine protease proprotein selected from Table S1, including biologically active variants and fragments thereof. In specific embodiments, the serine protease comprises, consists, or consists essentially of an amino acid sequence that is at least 80, 85, 90, 95, 98, or 100% identical to a sequence selected from Table S1.

In certain embodiments, the serine protease protein is composed of the active peptidase domain of a serine protease. Exemplary peptidase domain sequences of PPE (including exemplary mutants thereof), human ELANE, human CTSG, and a human PR3 are provided in Table S2 below.

TABLE S2
Exemplary Peptidase Domain Sequences

| | | SEQ ID |
|---|---|---|
| Name | Sequence | NO: |
| PPE | VVGGTEAQRNSWPSQISLQYRSGSSWAHTCGGTLIRQNWVMTAAHCVDRELT | 5 |
| Mutant F | FRVVVGEHNLNQNDGTEQYVGVQKIVVHPYWNTDDVAAGYDIALLRLAQSVT | |
| Q211F | LNSYVQLGVLPRAGTILANNSPCYITGWGLTRTNGQLAQTLQQAYLPTVDYA | |
| | ICSSSSYWGSTVKNSMVCAGGDGVRSGCFGDSGGPLHCLVNGQYAVHGVTSF | |
| | VSRLGCNVTRKPTVFTRVSAYISWINNVIASN | |
| PPE | VVGGTEAQRNSWPSQISLQYRSGSSWAHACGGTLIRQNWVMTAAHCVDRELT | 6 |
| Mutant H | FRVVVGEHNLNQNDGTEQYVGVQKIVVHPYWNTDDVAAGYDIALLRLAQSVT | |
| T55A | LNSYVQLGVLPRAGTILANNSPCYITGWGLTRTNGQLAQTLQQAYLPTVDYA | |
| | ICSSSSYWGSTVKNSMVCAGGDGVRSGCQGDSGGPLHCLVNGQYAVHGVTSF | |
| | VSRLGCNVTRKPTVFTRVSAYISWINNVIASN | |
| PPE | VVGGTEAQRNSWPSQISLQYRSGSSWAHACGGTLIRQNWVMTAAHCVDRELT | |
| Mutant F-H | FRVVVGEHNLNQNDGTEQYVGVQKIVVHPYWNTDDVAAGYDIALLRLAQSVT | |
| Q211F- | LNSYVQLGVLPRAGTILANNSPCYITGWGLTRTNGQLAQTLQQAYLPTVDYA | |
| T55A | ICSSSSYWGSTVKNSMVCAGGDGVRSGCFGDSGGPLHCLVNGQYAVHGVTSF | |
| | VSRLGCNVTRKPTVFTRVSAYISWINNVIASN | |
| PPE | VVGGTEAQRNSWPSQISLQYRSGSSWAHTCGGTLIRQNWVMTAAHCVDRELT | 8 |
| Mutant A | FRVVVGEHNLNQNDGTEQYVGVQKIVVHPYWNTDDVAAGYDIALLRLAQSVT | |
| N241A | LNSYVQLGVLPRAGTILANNSPCYITGWGLTRINGQLAQTLQQAYLPTVDYA | |
| | ICSSSSYWGSTVKNSMVCAGGDGVRSGCQGDSGGPLHCLVNGQYAVHGVTSF | |
| | VSRLGCAVTRKPTVFTRVSAYISWINNVIASN | |
| PPE | VVGGTEAQRNSWPSQISLQYRSGSSWAHTCGGTLIRQNWVMTAAHCVDRELT | 9 |
| Mutant B | FRVVVGEHNLNQNDGTEQYVGVQKIVVHPYWNTDDVAAGYDIALERLAQSVT | |
| N241Y | LNSYVQLGVLPRAGTILANNSPCYITGWGLTRTNGQLAQTLQQAYLPTVDYA | |
| | ICSSSSYWGSTVKNSMVCAGGDGVRSGCQGDSGGPLHCLVNGQYAVHGVTSF | |
| | VSRLGCYVTRKPTVFTRVSAYISWINNVIASN | |
| PPE | VVGGTEAQRNSWPSQISLQYRSGSSWAHTCGGTLIRQNWVMTAAHCVDAELT | 10 |
| Mutant C | FRVVVGEHNLNQNDGTEQYVGVQKIVVHPYWNTDDVAAGYDIALLRLAQSVT | |
| R75A | LNSYVQLGVLPRAGTILANNSPCYITGWGLTRINGQLAQTLQQAYLPTVDYA | |
| | ICSSSSYWGSTVKNSMVCAGGDGVRSGCQGDSGGPLHCLVNGQYAVHGVTSF | |
| | VSRLGCNVTRKPTVFTRVSAYISWINNVIASN | |
| PPE | VVGGTEAQRNSWPSQISLQYRSGSSWAHTCGGTLIRQNWVMTAAHCVDEELT | 11 |
| Mutant D | FRVVVGEHNLNQNDGTEQYVGVQKIVVHPYWNTDDVAAGYDIALLRLAQSVT | |
| R75E | LNSYVQLGVLPRAGTILANNSPCYITGWGLTRTNGQLAQTLQQAYLPTVDYA | |
| | ICSSSSYWGSTVKNSMVCAGGDGVRSGCQGDSGGPLHCLVNGQYAVHGVTSF | |
| | VSRLGCNVTRKPTVFTRVSAYISWINNVIASN | |
| PPE | VVGGTEAQRNSWPSQISLQYRSGSSWAHTCGGTLIRQNWVMTAAHCVDRELT | 12 |
| Mutant E | FRVVVGEHNLNQNDGTEQYVGVQKIVVHPYWNTDDVAAGYDIALLRLAQSVT | |
| Q211A | LNSYVQLGVLPRAGTILANNSPCYITGWGLTRINGQLAQTLQQAYLPTVDYA | |
| | ICSSSSYWGSTVKNSMVCAGGDGVRSGCAGDSGGPLHCLVNGQYAVHGVTSF | |
| | VSRLGCNVTRKPTVFTRVSAYISWINNVIASN | |
| PPE | VVGGTEAQRNSWPSQISLQYRSGSSWAHTCGGTLIRQNWVMTAAHCVDRELT | 13 |
| Mutant G | FRVVVGEHNLNQNDGTEQYVGVQKIVVHPYWNTDDVAAGYDIALLRLAQSVT | |
| R237A | LNSYVQLGVLPRAGTILANNSPCYITGWGLTRINGQLAQTLQQAYLPTVDYA | |
| | ICSSSSYWGSTVKNSMVCAGGDGVRSGCQGDSGGPLHCLVNGQYAVHGVTSF | |
| | VSALGCNVTRKPTVFTRVSAYISWINNVIASN | |
| PPE | VVGGTEAQRNSWPSQISLQYRSGSSWAHTCGGTLIRQNWVMTAAHCVDRELT | 14 |
| Mutant I | FRVVVGEHNLNQNDGTEQYVGVQKIVVHPYWNTDDVAAGYDIALLRLAQSVT | |
| S214A | LNSYVQLGVLPRAGTILANNSPCYITGWGLTRTNGQLAQTLQQAYLPTVDYA | |
| | ICSSSSYWGSTVKNSMVCAGGDGVRSGCQGDAGGPLHCLVNGQYAVHGVTSF | |
| | VSRLGCNVTRKPTVFTRVSAYISWINNVIASN | |
| PPE | VVGGTEAQRNSWPSQISLQYRSGSSWAHTCGGTLIRQNWVMTAAHCVARELT | 15 |
| Mutant J | FRVVVGEHNLNQNDGTEQYVGVQKIVVHPYWNTDDVAAGYDIALLRLAQSVT | |
| D74A | LNSYVQLGVLPRAGTILANNSPCYITGWGLTRTNGQLAQTLQQAYLPTVDYA | |
| | ICSSSSYWGSTVKNSMVCAGGDGVRSGCQGDSGGPLHCLVNGQYAVHGVTSF | |
| | VSRLGCNVTRKPTVFTRVSAYISWINNVIASN | |
| WT PPE | VVGGTEAQRNSWPSQISLQYRSGSSWAHTCGGTLIRQNWVMTAAHCVDRELT | 16 |
| | FRVVVGEHNLNQNDGTEQYVGVQKIVVHPYWNTDDVAAGYDIALERLAQSVT | |
| | LNSYVQLGVLPRAGTILANNSPCYITGWGLTRINGQLAQTLQQAYLPTVDYA | |
| | ICSSSSYWGSTVKNSMVCAGGDGVRSGCQGDSGGPLHCLVNGQYAVHGVTSF | |
| | VSRLGCNVTRKPTVFTRVSAYISWINNVIASN | |
| Human | IVGGRRARPHAWPFMVSLQLRGGHFCGATLIAPNFVMSAAHCVANVNVRAVR | 17 |
| ELANE | VVLGAHNLSRREPTRQVFAVQRIFENGYDPVNLLNDIVILQLNGSATINANV | |
| | QVAQLPAQGRRLGNGVQCLAMGWGLLGRNRGIASVLQELNVTVVTSLCRRSN | |
| | VCTLVRGRQAGVCFGDSGSPLVCNGLIHGIASFVRGGCASGLYPDAFAPVAQ | |
| | FVNWIDSIIQRSEDNPCPHPRDPDPASRTH | |
| Human CTSG | IIGGRESRPHSRPYMAYLQIQSPAGQSRCGGFLVREDFVLTAAHCWGSNINV | 18 |
| | TLGAHNIQRRENTQQHITARRAIRHPQYNQRTIQNDIMLLQLSRRVRRNRNV | |
| | NPVALPRAQEGLRPGTLCTVAGWGRVSMRRGTDTLREVQLRVQRDRQCLRIF | |
| | GSYDPRRQICVGDRRERKAAFKGDSGGPLLCNNVAHGIVSYGKSSGVPPEVF | |
| | TRVSSFLPWIRTTMRSFKLLDQMETPL | |
| Human PR3 | IVGGHEAQPHSRPYMASLQMRGNPGSHFCGGTLIHPSFVLTAAHCLRDIPQR | 19 |
| | LVNVVLGAHNVRTQEPTQQHFSVAQVFLNNYDAENKINDVLLIQLSSPANLS | |
| | ASVATVQLPQQDQPVPHGTQCLAMGWGRVGAHDPPAQVLQELNVTVVTFFCR | |
| | PHNICTFVPRRKAGICFGDSGGPLICDGIIQGIDSFVIWGCATRLFPDFFTR | |
| | VALYVDWIRSTLRRVEAKGRP | |
| Human | IIGGHEAKPHSRPYMAYLMIWDQKSLKRCGGFLIRDDFVLTAAHCWGSSINV | 20 |
| granzyme B | TLGAHNIKEQEPTQQFIPVKRPIPHPAYNPKNFSNDIMLLQLERKAKRTRAV | |
| | QPLRLPSNKAQVKPGQTCSVAGWGQTAPLGKHSHTLQEVKMTVQEDRKCESD | |
| | LRHYYDSTIELCVGDPEIKKTSFKGDSGGPLVCNKVAQGIVSYGRNNGMPPR | |
| | ACTKVSSFVHWIKKTMKRY | |

Thus, in some embodiments, the serine protease protein comprises, consists, or consists essentially of a serine protease peptidase domain sequence selected from Table S2, including biologically active variants and fragments thereof. In specific embodiments, the serine protease protein comprises, consists, or consists essentially of an amino acid sequence that is at least 80, 85, 90, 95, 98, or 100% identical to a sequence selected from Table S2.

In some embodiments, the serine protease protein is a PPE protein, for example, wherein:
the PPE protein comprises, consists, or consists essentially of an amino acid sequence that is at least 80, 85, 90, 95, 98, 99, or 100% identical to SEQ ID NO: 5, and which retains the Q211F amino acid substitution;
the PPE protein comprises, consists, or consists essentially of an amino acid sequence that is at least 80, 85, 90, 95, 98, 99, or 100% identical to SEQ ID NO: 6, and which retains the T55A amino acid substitution;
the PPE protein comprises, consists, or consists essentially of an amino acid sequence that is at least 80, 85, 90, 95, 98, 99, or 100% identical to SEQ ID NO: 7, and which retains the Q211F and T55A amino acid substitutions;
the PPE protein comprises, consists, or consists essentially of an amino acid sequence that is at least 80, 85, 90, 95, 98, 99, or 100% identical to SEQ ID NO: 8, and which retains the N241A amino acid substitution;
the PPE protein comprises, consists, or consists essentially of an amino acid sequence that is at least 80, 85, 90, 95, 98, 99, or 100% identical to SEQ ID NO: 9, and which retains the N241Y amino acid substitution;
the PPE protein comprises, consists, or consists essentially of an amino acid sequence that is at least 80, 85, 90, 95, 98, 99, or 100% identical to SEQ ID NO: 10, and which retains the R75A amino acid substitution;
the PPE protein comprises, consists, or consists essentially of an amino acid sequence that is at least 80, 85, 90, 95, 98, 99, or 100% identical to SEQ ID NO: 11, and which retains the R75E amino acid substitution;
the PPE protein comprises, consists, or consists essentially of an amino acid sequence that is at least 80, 85, 90, 95, 98, 99, or 100% identical to SEQ ID NO: 12, and which retains the Q211A amino acid substitution;
the PPE protein comprises, consists, or consists essentially of an amino acid sequence that is at least 80, 85, 90, 95, 98, 99, or 100% identical to SEQ ID NO: 13, and which retains the R237A amino acid substitution;
the PPE protein comprises, consists, or consists essentially of an amino acid sequence that is at least 80, 85, 90, 95, 98, 99, or 100% identical to SEQ ID NO: 14, and which retains the S214A amino acid substitution;
the PPE protein comprises, consists, or consists essentially of an amino acid sequence that is at least 80, 85, 90, 95, 98, 99, or 100% identical to SEQ ID NO: 15, and which retains the D74A amino acid substitution; and
the PPE protein comprises, consists, or consists essentially of an amino acid sequence that is at least 80, 85, 90, 95, 98, 99, or 100% identical to SEQ ID NO: 16.

In some embodiments, the serine protease protein is a human ELANE protein, for example, wherein the human ELANE protein comprises, consists, or consists essentially of an amino acid sequence that is at least 80, 85, 90, 95, 98, 99, or 100% identical to SEQ ID NO: 17. In some embodiments, the serine protease protein is a human CTSG protein, for instance, wherein the human CTSG protein comprises, consists, or consists essentially of an amino acid sequence that is at least 80, 85, 90, 95, 98, 99, or 100% identical to SEQ ID NO: 18. In some embodiments, the serine protease protein is a human PR3 protein, for example, wherein the human PR3 protein comprises, consists, or consists essentially of an amino acid sequence that is at least 80, 85, 90, 95, 98, 99, or 100% identical to SEQ ID NO: 19. In some embodiments, the serine protease protein is a human granzyme B protein, for example, wherein the human granzyme B protein comprises, consists, or consists essentially of an amino acid sequence that is at least 80, 85, 90, 95, 98, 99, or 100% identical to SEQ ID NO: 20.

As noted above, in certain embodiments, (a) and (b) are present in the composition at a molar ratio of [(a):(b)] that ranges from about 1:3 to about 1:1, for example, a molar ratio of about 1:3, 1:2.9, 1:2.8, 1:2.7, 1:2.6, 1:2.5, 1:2.4, 1:2.3, 1:2.1, 1:2, 1:1.9, 1:1.8, 1:1.7, 1:1.6, 1:1.5, 1:1.4, 1:1.3, 1:1.2, 1:1.1, or 1:1. In specific aspects, the molar ratio defined herein retains the CD95 protease cleavage and cancer cell-killing activity of the serine protease while sterically hindering it from binding to serine protease inhibitors (e.g., A1AT), fibrinogen and/or plasma antibodies, thereby protecting it from inhibition by serine protease inhibitors or plasma antibodies, and inhibiting or otherwise reducing its ability to cleave fibrinogen.

Thus, in certain embodiments, the protein complex retains the ability to cleave CD95 (Fas Receptor) and does not substantially cleave fibrinogen. In some embodiments, a protein complex described herein has about or at least about 50, 60, 70, 80, 90, 100, 200, 300, 400, 500, 600, 700, 800, 900, or 1000% or more of the CD95 protease cleavage and/or cancer cell-killing activity of the corresponding serine protease protein on its own (for example, in the presence of serine protease inhibitors in plasma such as ALAT). In certain embodiments, a protein complex described herein has about or less than about 50, 40, 30, 20, 10, 5% or less of the fibrinogen protease cleavage activity of the corresponding serine protease protein on its own. CD95 cleavage activity, cancer cell-killing activity, fibrinogen cleavage activity, and coagulation properties can be measured according to routine techniques in the art (see, the Examples). For example, serine protease activity more generally can be monitored using a colorimetric substrate activity assay (N-Methoxysuccinyl-Ala-Ala-Pro-Val p-nitroanilide), and CD95 and/or fibrinogen cleavage can be measured directly (e.g., Western blot). As desired, protease cleavage activity can be measured in the presence of serine protease inhibitors such as ALAT. Cancer cell-killing activity can be measured in vitro or in vivo, and effects on coagulation in vivo can be measured, for example, by routine assays such as prothrombin (PT) time and partial thromboplastin (PTT) time (see the Examples).

In some embodiments, a protein complex described herein is generated in vivo or ex vivo, for example, in a cell by contacting a cell or subject with one or more expressible polynucleotides that encode the (a) alpha-2-macroglobulin (A2M) proteins and the (b) serine protease proteins. The protein complexes then form within the cell. An “expressible polynucleotide” includes a DNA, cDNA, RNA, mRNA or other polynucleotide that comprises at least one coding sequence for (a) and/or (b) and optionally at least one expression control sequence, for example, a transcriptional and/or translational regulatory element, and which can express the encoded protein(s) upon introduction into the cell, for example, a cell in the subject. Certain embodiments include contacting an ex vivo cell with the one or more expressible polynucleotides that encode (a) and (b) and administering the cell to a subject.

Exemplary viral vectors that can be utilized to deliver an expressible polynucleotide include adenoviral vectors, herpes virus vectors, vaccinia virus vectors, adeno-associated virus (AAV) vectors, and retroviral vectors such as lentiviral vectors. Examples of retroviral vectors include, but are not limited to Moloney murine leukemia virus (MoMuL V), Harvey murine sarcoma virus (HaMuSV), murine mammary tumor virus (MuMTV), SIV, BIV, HIV and Rous Sarcoma Virus (RSV)-based vectors. In particular embodiments, the expressible polynucleotide is a modified RNA or modified mRNA polynucleotide, for example, a non-naturally occurring RNA analog. In certain embodiments, the modified RNA or mRNA polypeptide comprises one or more modified or non-natural bases. In some embodiments, the modified mRNA comprises one or more modified or non-natural internucleotide linkages. Expressible RNA polynucleotides for delivering an encoded protein are described, for example, in Kormann et al., Nat Biotechnol. 29:154-7, 2011; and U.S. Application Nos. 2015/0111248; 2014/0243399; 2014/0147454; and 2013/0245104, which are incorporated by reference in their entireties.

In some embodiments, a protein complex described herein has one or more improved biological, physical, and/or pharmacokinetic properties, relative to the corresponding serine protease protein on its own. The protein complexes described herein can be used in any of the compositions, methods, and/or kits described herein.

### Methods of Use and Pharmaceutical Compositions

Certain embodiments include methods of treating, ameliorating the symptoms of, and/or reducing the progression of, a disease or condition in a subject in need thereof, comprising administering to the subject a composition comprising a protein complex, as described herein. In particular embodiments, the disease is a cancer, that is, the subject in need thereof has, is suspected of having, or is at risk for having, a cancer.

In particular embodiments, the cancer is a primary cancer or a metastatic cancer. In specific embodiments, the cancer is selected from one or more of melanoma (optionally metastatic melanoma), breast cancer (optionally triple-negative breast cancer, TNBC), kidney cancer (optionally renal cell carcinoma), pancreatic cancer, bone cancer, prostate cancer, lung cancer (for example, small cell lung cancer, non-small cell lung cancer (NSCLC), squamous cell lung carcinoma), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

In some embodiments, as noted above, the cancer is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

In certain embodiments, administration (for example, intravenous administration) of the serine protease-containing protein complex or composition does not substantially impact coagulation in the subject, for example, it does not substantially increase prothrombin time or partial thromboplastin time in the subject (for example, relative to administration of the corresponding serine protease on its own). In certain embodiments, administration of a serine protease-containing protein complex described herein has a significantly reduced impact on coagulation in the subject relative to administration of the corresponding serine protease alone.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Certain embodiments thus include combination therapies for treating cancers, including methods of treating ameliorating the symptoms of, or inhibiting the progression of, a cancer in a subject in need thereof, comprising administering to the subject a composition of a protein complex described herein in combination with at least one additional agent, for example, an immunotherapy agent, a chemotherapeutic agent, a hormonal therapeutic agent, and/or a kinase inhibitor. In some embodiments, administering the composition enhances the susceptibility of the cancer to the additional agent (for example, immunotherapy agent, chemotherapeutic agent, hormonal therapeutic agent, and or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone.

Certain combination therapies employ one or more cancer immunotherapy agents, or “immunotherapy agents”. In certain instances, an immunotherapy agent modulates the immune response of a subject, for example, to increase or maintain a cancer-related or cancer-specific immune response, and thereby results in increased immune cell inhibition or reduction of cancer cells. Exemplary immunotherapy agents include polypeptides, for example, antibodies and antigen-binding fragments thereof, ligands, and small peptides, and mixtures thereof. Also include as immunotherapy agents are small molecules, cells (e.g., immune cells such as T-cells), various cancer vaccines, gene therapy or other polynucleotide-based agents, including viral agents such as oncolytic viruses, and others known in the art. Thus, in certain embodiments, the cancer immunotherapy agent is selected from one or more of immune checkpoint modulatory agents, cancer vaccines, oncolytic viruses, cytokines, and cell-based immunotherapies.

In certain embodiments, the cancer immunotherapy agent is an immune checkpoint modulatory agent. Particular examples include “antagonists” of one or more inhibitory immune checkpoint molecules, and “agonists” of one or more stimulatory immune checkpoint molecules. Generally, immune checkpoint molecules are components of the immune system that either turn up a signal (co-stimulatory molecules) or turn down a signal, the targeting of which has therapeutic potential in cancer because cancer cells can perturb the natural function of immune checkpoint molecules (see, e.g., Sharma and Allison, Science. 348:56-61, 2015; Topalian et al., Cancer Cell. 27:450-461, 2015; Pardoll, Nature Reviews Cancer. 12:252-264, 2012). In some embodiments, the immune checkpoint modulatory agent (e.g., antagonist, agonist) “binds” or “specifically binds” to the one or more immune checkpoint molecules, as described herein.

In some embodiments, the immune checkpoint modulatory agent is an antagonist or inhibitor of one or more inhibitory immune checkpoint molecules. Exemplary inhibitory immune checkpoint molecules include Programmed Death-Ligand 1 (PD-L1), Programmed Death-Ligand 2 (PD-L2), Programmed Death 1 (PD-1), V-domain Ig suppressor of T cell activation (VISTA), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), B and T Lymphocyte Attenuator (BTLA), CD160, and T-cell immunoreceptor with Ig and ITIM domains (TIGIT).

In certain embodiments, the agent is a PD-1 (receptor) antagonist or inhibitor, the targeting of which has been shown to restore immune function in the tumor environment (see, e.g., Phillips et al., Int Immunol. 27:39-46, 2015). PD-1 is a cell surface receptor that belongs to the immunoglobulin superfamily and is expressed on T cells and pro-B cells. PD-1 interacts with two ligands, PD-L1 and PD-L2. PD-1 functions as an inhibitory immune checkpoint molecule, for example, by reducing or preventing the activation of T-cells, which in turn reduces autoimmunity and promotes self-tolerance. The inhibitory effect of PD-1 is accomplished at least in part through a dual mechanism of promoting apoptosis in antigen specific T-cells in lymph nodes while also reducing apoptosis in regulatory T cells (suppressor T cells). Some examples of PD-1 antagonists or inhibitors include an antibody or antigen-binding fragment or small molecule that specifically binds to PD-1 and reduces one or more of its immune-suppressive activities, for example, its downstream signaling or its interaction with PD-L1. Specific examples of PD-1 antagonists or inhibitors include the antibodies nivolumab, pembrolizumab, PDR001, MK-3475, AMP-224, AMP-514, and pidilizumab, and antigen-binding fragments thereof (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579).

In some embodiments, the agent is a PD-L1 antagonist or inhibitor. As noted above, PD-L1 is one of the natural ligands for the PD-1 receptor. General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MEDI4736), and antigen-binding fragments thereof (see, e.g., U.S. Pat. Nos. 9,102,725; 9,393,301; 9,402,899; 9,439,962).

In some embodiments, the agent is a PD-L2 antagonist or inhibitor. As noted above, PD-L2 is one of the natural ligands for the PD-1 receptor. General examples of PD-L2 antagonists or inhibitors include an antibody or antigen-binding fragment or small molecule that specifically binds to PD-L2 and reduces one or more of its immune-suppressive activities, for example, its binding to the PD-1 receptor.

In certain embodiments, the agent is a VISTA antagonist or inhibitor. VISTA is approximately 50 kDa in size and belongs to the immunoglobulin superfamily (it has one IgV domain) and the B7 family. It is primarily expressed in white blood cells, and its transcription is partially controlled by p53. There is evidence that VISTA can act as both a ligand and a receptoron T cells to inhibit T cell effector function and maintain peripheral tolerance. VISTA is produced at high levels in tumor-infiltrating lymphocytes, such as myeloid-derived suppressor cells and regulatory T cells, and its blockade with an antibody results in delayed tumor growth in mouse models of melanoma and squamous cell carcinoma. Exemplary anti-VISTA antagonist antibodies include, for example, the antibodies described in WO 2018/237287, which is incorporated by reference in its entirety.

In some embodiments, the agent is a CTLA-4 antagonist or inhibitor. CTLA4 or CTLA-4 (cytotoxic T-lymphocyte-associated protein 4), also known as CD152 (cluster of differentiation 152), is a protein receptor that functions as an inhibitory immune checkpoint molecule, for example, by transmitting inhibitory signals to T-cells when it is bound to CD80 or CD86 on the surface of antigen-presenting cells. General examples CTLA-4 antagonists or inhibitors include an antibody or antigen-binding fragment or small molecule that specifically binds to CTLA-4. Particular examples include the antibodies ipilimumab and tremelimumab, and antigen-binding fragments thereof. At least some of the activity of ipilimumab is believed to be mediated by antibody-dependent cell-mediated cytotoxicity (ADCC) killing of suppressor Tregs that express CTLA-4.

In some embodiments, the agent is an IDO antagonist or inhibitor, or a TDO antagonist or inhibitor. IDO and TDO are tryptophan catabolic enzymes with immune-inhibitory properties. For example, IDO is known to suppress T-cells and NK cells, generate and activate Tregs and myeloid-derived suppressor cells, and promote tumor angiogenesis. General examples of IDO and TDO antagonists or inhibitors include an antibody or antigen-binding fragment or small molecule that specifically binds to IDO or TDO (see, e.g., Platten et al., Front Immunol. 5:673, 2014) and reduces or inhibits one or more immune-suppressive activities. Specific examples of IDO antagonists or inhibitors include indoximod (NLG-8189), 1-methyl-tryptophan (IMT), β-Carboline (norharmane; 9H-pyrido[3,4-b]indole), rosmarinic acid, and epacadostat (see, e.g., Sheridan, Nature Biotechnology. 33:321-322, 2015). Specific examples of TDO antagonists or inhibitors include 680C91 and LM10 (see, e.g., Pilotte et al., PNAS USA. 109:2497-2502, 2012).

In some embodiments, the agent is a TIM-3 antagonist or inhibitor. T-cell Immunoglobulin domain and Mucin domain 3 (TIM-3) is expressed on activated human CD4+ T-cells and regulates Th1 and Th17 cytokines. TIM-3 also acts as a negative regulator of Th1/Tc1 function by triggering cell death upon interaction with its ligand, galectin-9. TIM-3 contributes to the suppressive tumor microenvironment and its overexpression is associated with poor prognosis in a variety of cancers (see, e.g., Li et al., Acta Oncol. 54:1706-13, 2015). General examples of TIM-3 antagonists or inhibitors include an antibody or antigen-binding fragment or small molecule that specifically binds to TIM-3 and reduces or inhibits one or more of its immune-suppressive activities.

In some embodiments, the agent is a LAG-3 antagonist or inhibitor. Lymphocyte Activation Gene-3 (LAG-3) is expressed on activated T-cells, natural killer cells, B-cells and plasmacytoid dendritic cells. It negatively regulates cellular proliferation, activation, and homeostasis of T-cells, in a similar fashion to CTLA-4 and PD-1 (see, e.g., Workman and Vignali, European Journal of Immun. 33:970-9, 2003; and Workman et al., Journal of Immun. 172:5450-5, 2004), and has been reported to play a role in Treg suppressive function (see, e.g., Huang et al., Immunity. 21:503-13, 2004). LAG3 also maintains CD8+ T-cells in a tolerogenic state and combines with PD-1 to maintain CD8 T-cell exhaustion. General examples of LAG-3 antagonists or inhibitors include an antibody or antigen-binding fragment or small molecule that specifically binds to LAG-3 and inhibits one or more of its immune-suppressive activities. Specific examples include the antibody BMS-986016, and antigen-binding fragments thereof.

In some embodiments, the agent is a BTLA antagonist or inhibitor. B- and T-lymphocyte attenuator (BTLA; CD272) expression is induced during activation of T-cells, and it inhibits T-cells via interaction with tumor necrosis family receptors (TNF-R) and B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses, for example, by inhibiting the function of human CD8+ cancer-specific T-cells (see, e.g., Derré et al., J Clin Invest 120:157-67, 2009). General examples of BTLA antagonists or inhibitors include an antibody or antigen-binding fragment or small molecule that specifically binds to BTLA-4 and reduce one or more of its immune-suppressive activities.

In some embodiments, the agent is an HVEM antagonist or inhibitor, for example, an antagonist or inhibitor that specifically binds to HVEM and interferes with its interaction with BTLA or CD160. General examples of HVEM antagonists or inhibitors include an antibody or antigen-binding fragment or small molecule that specifically binds to HVEM, optionally reduces the HVEM/BTLA and/or HVEM/CD160 interaction, and thereby reduces one or more of the immune-suppressive activities of HVEM.

In some embodiments, the agent is a CD160 antagonist or inhibitor, for example, an antagonist or inhibitor that specifically binds to CD160 and interferes with its interaction with HVEM. General examples of CD160 antagonists or inhibitors include an antibody or antigen-binding fragment or small molecule that specifically binds to CD160, optionally reduces the CD160/HVEM interaction, and thereby reduces or inhibits one or more of its immune-suppressive activities.

In some embodiments, the agent is a TIGIT antagonist or inhibitor. T cell Ig and ITIM domain (TIGIT) is a co-inhibitory receptor that is found on the surface of a variety of lymphoid cells, and suppresses antitumor immunity, for example, via Tregs (Kurtulus et al., J Clin Invest. 125:4053-4062, 2015). General examples of TIGIT antagonists or inhibitors include an antibody or antigen-binding fragment or small molecule that specifically binds to TIGIT and reduce one or more of its immune-suppressive activities (see, e.g., Johnston et al., Cancer Cell. 26:923-37, 2014).

In certain embodiments, the immune checkpoint modulatory agent is an agonist of one or more stimulatory immune checkpoint molecules. Exemplary stimulatory immune checkpoint molecules include CD40, OX40, Glucocorticoid-Induced TNFR Family Related Gene (GITR), CD137 (4-1BB), CD27, CD28, CD226, and Herpes Virus Entry Mediator (HVEM).

In some embodiments, the agent is a CD40 agonist. CD40 is expressed on antigen-presenting cells (APC) and some malignancies. Its ligand is CD40L (CD154). On APC, ligation results in upregulation of costimulatory molecules, potentially bypassing the need for T-cell assistance in an antitumor immune response. CD40 agonist therapy plays an important role in APC maturation and their migration from the tumor to the lymph nodes, resulting in elevated antigen presentation and T cell activation. Anti-CD40 agonist antibodies produce substantial responses and durable anticancer immunity in animal models, an effect mediated at least in part by cytotoxic T-cells (see, e.g., Johnson et al. Clin Cancer Res. 21:1321-1328, 2015; and Vonderheide and Glennie, Clin Cancer Res. 19:1035-43, 2013). General examples of CD40 agonists include an antibody or antigen-binding fragment or small molecule or ligand that specifically binds to CD40 and increases one or more of its immunostimulatory activities. Specific examples include CP-870,893, dacetuzumab, Chi Lob 7/4, ADC-1013, CD40L, rbCD40L, and antigen-binding fragments thereof. Specific examples of CD40 agonists include, but are not limited to, APX005 (see, e.g., US 2012/0301488) and APX005M (see, e.g., US 2014/0120103).

In some embodiments, the agent is an OX40 agonist. OX40 (CD134) promotes the expansion of effector and memory T cells, and suppresses the differentiation and activity of T-regulatory cells (see, e.g., Croft et al., Immunol Rev. 229:173-91, 2009). Its ligand is OX40L (CD252). Since OX40 signaling influences both T-cell activation and survival, it plays a key role in the initiation of an anti-tumor immune response in the lymph node and in the maintenance of the anti-tumor immune response in the tumor microenvironment. General examples of OX40 agonists include an antibody or antigen-binding fragment or small molecule or ligand that specifically binds to OX40 and increases one or more of its immunostimulatory activities. Specific examples include OX86, OX-40L, Fc-OX40L, GSK3174998, MEDI0562 (a humanized OX40 agonist), MEDI6469 (murine OX4 agonist), and MEDI6383 (an OX40 agonist), and antigen-binding fragments thereof.

In some embodiments, the agent is a GITR agonist. Glucocorticoid-Induced TNFR family Related gene (GITR) increases T cell expansion, inhibits the suppressive activity of Tregs, and extends the survival of T-effector cells. GITR agonists have been shown to promote an anti-tumor response through loss of Treg lineage stability (see, e.g., Schaer et al., Cancer Immunol Res. 1:320-31, 2013). These diverse mechanisms show that GITR plays an important role in initiating the immune response in the lymph nodes and in maintaining the immune response in the tumor tissue. Its ligand is GITRL. General examples of GITR agonists include an antibody or antigen-binding fragment or small molecule or ligand that specifically binds to GITR and increases one or more of its immunostimulatory activities. Specific examples include GITRL, INCAGN01876, DTA-1, MEDI1873, and antigen-binding fragments thereof.

In some embodiments, the agent is a CD137 agonist, CD137 (4-1BB) is a member of the tumor necrosis factor (TNF) receptor family, and crosslinking of CD137 enhances T-cell proliferation, IL-2 secretion, survival, and cytolytic activity. CD137-mediated signaling also protects T-cells such as CD8+ T-cells from activation-induced cell death. General examples of CD137 agonists include an antibody or antigen-binding fragment or small molecule or ligand that specifically binds to CD137 and increases one or more of its immunostimulatory activities. Specific examples include the CD137 (or 4-1BB) ligand (see, e.g., Shao and Schwarz, J Leukoc Biol. 89:21-9, 2011) and the antibody utomilumab, including antigen-binding fragments thereof.

In some embodiments, the agent is a CD27 agonist. Stimulation of CD27 increases antigen-specific expansion of naïve T cells and contributes to T-cell memory and long-term maintenance of T-cell immunity. Its ligand is CD70. The targeting of human CD27 with an agonist antibody stimulates T-cell activation and antitumor immunity (see, e.g., Thomas et al., Oncoimmunology. 2014; 3: e27255. doi: 10.4161/onci.27255; and He et al., J Immunol. 191:4174-83, 2013). General examples of CD27 agonists include an antibody or antigen-binding fragment or small molecule or ligand that specifically binds to CD27 and increases one or more of its immunostimulatory activities. Specific examples include CD70 and the antibodies varlilumab and CDX-1127 (IF5), including antigen-binding fragments thereof.

In some embodiments, the agent is a CD28 agonist. CD28 is constitutively expressed CD4+ T cells some CD8+ T cells. Its ligands include CD80 and CD86, and its stimulation increases T-cell expansion. General examples of CD28 agonists include an antibody or antigen-binding fragment or small molecule or ligand that specifically binds to CD28 and increases one or more of its immunostimulatory activities. Specific examples include CD80, CD86, the antibody TAB08, and antigen-binding fragments thereof.

In some embodiments, the agent is CD226 agonist. CD226 is a stimulating receptor that shares ligands with TIGIT, and opposite to TIGIT, engagement of CD226 enhances T-cell activation (see, e.g., Kurtulus et al., J Clin Invest. 125:4053-4062, 2015; Bottino et al., J Exp Med. 1984:557-567, 2003; and Tahara-Hanaoka et al., Int Immunol. 16:533-538, 2004). General examples of CD226 agonists include an antibody or antigen-binding fragment or small molecule or ligand (e.g., CD112, CD155) that specifically binds to CD226 and increases one or more of its immunostimulatory activities.

In some embodiments, the agent is an HVEM agonist. Herpesvirus entry mediator (HVEM), also known as tumor necrosis factor receptor superfamily member 14 (TNFRSF14), is a human cell surface receptor of the TNF-receptor superfamily. HVEM is found on a variety of cells including T-cells, APCs, and other immune cells. Unlike other receptors, HVEM is expressed at high levels on resting T-cells and down-regulated upon activation. It has been shown that HVEM signaling plays a crucial role in the early phases of T-cell activation and during the expansion of tumor-specific lymphocyte populations in the lymph nodes. General examples of HVEM agonists include an antibody or antigen-binding fragment or small molecule or ligand that specifically binds to HVEM and increases one or more of its immunostimulatory activities.

In certain embodiments, the immunotherapy agent is a bi-specific or multi-specific antibody. For instance, certain bi-specific or multi-specific antibodies are able to (i) bind to and inhibit one or more inhibitory immune checkpoint molecules, and also (ii) bind to and agonize one or more stimulatory immune checkpoint molecules. In certain embodiments, a bi-specific or multi-specific antibody (i) binds to and inhibits one or more of PD-L1, PD-L2, PD-1, CTLA-4, IDO, TDO, TIM-3, LAG-3, BTLA, CD160, and/or TIGIT, and also (ii) binds to and agonizes one or more of CD40, OX40 Glucocorticoid-Induced TNFR Family Related Gene (GITR), CD137 (4-1BB), CD27, CD28, CD226, and/or Herpes Virus Entry Mediator (HVEM).

In some embodiments, the immunotherapy agent is a cancer vaccine. In certain embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge). In some embodiments, the cancer vaccine comprises or expresses a TAA or TSA as described herein.

In some embodiments, the immunotherapy agent is an oncolytic viruses. In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

In certain embodiments, the cancer immunotherapy agent is a cytokine. Exemplary cytokines include interferon (IFN)-α, IL-2, IL-12, IL-7, IL-21, and Granulocyte-macrophage colony-stimulating factor (GM-CSF).

In certain embodiments, the cancer immunotherapy agent is cell-based immunotherapy, for example, a therapy that utilizes immune cells, including ex vivo-derived immune cells, such as lymphocytes, natural killer (NK) cells, macrophages, and/or dendritic cells (DCs). In some embodiments, the lymphocytes comprise T-cells, for example, cytotoxic T-lymphocytes (CTLs). See, for example, June, J Clin Invest. 117:1466-1476, 2007; Rosenberg and Restifo, Science. 348:62-68, 2015; Cooley et al., Biol. of Blood and Marrow Transplant. 13:33-42, 2007; and Li and Sun, Chin J Cancer Res. 30:173-196, 2018, for descriptions of adoptive T-cell and NK cell immunotherapies. In some embodiments, the T-cells comprise cancer antigen-specific T-cells, which are directed against at least one cancer antigen. In some embodiments, the cancer antigen-specific T-cells are selected from one or more of chimeric antigen receptor (CAR)-modified T-cells, T-cell Receptor (TCR)-modified T-cells, tumor infiltrating lymphocytes (TILs), and peptide-induced T-cells. In specific embodiments, the CAR-modified T-cell is targeted against CD-19 (see, e.g., Maude et al., Blood. 125:4017-4023, 2015). In some instances, the ex vivo-derived immune cells are autologous cells, which are obtained from the patient to be treated.

Certain combination therapies employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type 1 or type II), and anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine).

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The various chemotherapeutic agents described herein can be combined with any one or more of the protein complexes described herein, and used according to any one or more of the methods or compositions described herein.

Certain combination therapies employ at least one hormonal therapeutic agent, General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH) s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

The various hormonal therapeutic agents described herein can be combined with any one or more of the protein complexes described herein, and used according to any one or more of the methods or compositions described herein.

Certain combination therapies employ at least one kinase inhibitor, including tyrosine kinase inhibitors. Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib.

The various kinase inhibitors described herein can be combined with any one or more of the protein complexes described herein, and used according to any one or more of the methods or compositions described herein.

In some embodiments, the methods and compositions described herein increase cancer cell-killing in the subject by about or at least about 2-fold, 5-fold, 10-fold, 50-fold, 100-fold, 500-fold, or 1000-fold or more relative to a control or reference. In some embodiments, the methods and compositions described herein increase an immune response in the subject by about or at least about 5, 10, 15, 20, 25, 30, 35, 40, 45, 50, 60, 70, 80, 90, 100, 200, 300, 400, 500, 600, 700, 800, 900, 1000, 2000% or more, or by about 2-fold, 5-fold, 10-fold, 50-fold, 100-fold, 500-fold, or 1000-fold or more, relative to a control or reference (e.g., relative to a corresponding serine protease on its own), including wherein the immune response is an anti-cancer immune response.

In some embodiments, the methods and compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and compositions described herein increase median survival time of a subject by 1 year, 2 years, 3 years, or longer. In some embodiments, the methods and pharmaceutical compositions increase progression-free survival by 2 weeks, 3 weeks, 4 weeks, 5 weeks, 6 weeks, 7 weeks, 8 weeks, 9 weeks, 10 weeks or longer. In certain embodiments, the methods and pharmaceutical compositions described herein increase progression-free survival by 1 year, 2 years, 3 years, or longer.

In certain embodiments, the methods and compositions described herein are sufficient to result in tumor regression, for example, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and compositions described herein are sufficient to result in stable disease. In certain embodiments, the methods and compositions described herein are sufficient to result in clinically relevant reduction in symptoms of a particular disease indication known to the skilled clinician.

For in vivo use, as noted above, for the treatment of human or non-human mammalian disease or testing, the protein complexes described herein are generally incorporated into one or more therapeutic or pharmaceutical compositions prior to administration, including veterinary therapeutic compositions.

Thus, certain embodiments relate to pharmaceutical or therapeutic compositions that comprise a protein complex, as described herein. In some instances, a pharmaceutical or therapeutic composition comprises one or more of the protein complexes described herein in combination with a pharmaceutically- or physiologically-acceptable carrier or excipient. Certain pharmaceutical or therapeutic compositions further comprise at least one additional agent, for example, an immunotherapy agent, a chemotherapeutic agent, a hormonal therapeutic agent, and/or a kinase inhibitor as described herein.

In particular embodiments, the pharmaceutical or therapeutic compositions comprising a protein complex is substantially pure on a protein basis or a weight-weight basis, for example, the composition has a purity of at least about 80%, 85%, 90%, 95%, 98%, or 99% on a protein basis or a weight-weight basis.

In some embodiments, the protein complexes described herein do not form aggregates, have a desired solubility, and/or have an immunogenicity profile that is suitable for use in humans, as known in the art. Thus, in some embodiments, a pharmaceutical or therapeutic composition comprising a protein complex is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2% high molecular weight aggregated proteins, or less than about 1% high molecular weight aggregated proteins.

In some embodiments, the protein complexes are concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11, 12, 13, 14 or 15 mg/ml and are formulated for biotherapeutic uses.

To prepare a therapeutic or pharmaceutical composition, an effective or desired amount of one or more protein complexes is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of protein complexes described herein, in pure form or in an appropriate therapeutic or pharmaceutical composition, can be carried out via any of the accepted modes of administration of agents for serving similar utilities. The therapeutic or pharmaceutical compositions can be prepared by combining a protein complex-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous, or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

The therapeutic or pharmaceutical compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In some instances, a therapeutically effective daily dose is (for a 70 kg mammal) from about 0.001 mg/kg (i.e., ~0.07 mg) to about 100 mg/kg (i.e., ~7.0 g); preferably a therapeutically effective dose is (for a 70 kg mammal) from about 0.01 mg/kg (i.e., ~0.7 mg) to about 50 mg/kg (i.e., ~3.5 g); more preferably a therapeutically effective dose is (for a 70 kg mammal) from about 1 mg/kg (i.e., ~70 mg) to about 25 mg/kg (i.e., ~1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains a protein complex and an additional therapeutic agent (e.g., immunotherapy agent, chemotherapeutic agent, hormonal therapeutic agent, kinase inhibitor), as well as administration of compositions comprising a protein complex and an additional therapeutic agent in its own separate pharmaceutical dosage formulation. For example, a protein complex and an additional therapeutic agent can be administered to the subject together in a single parenteral dosage composition such as in a saline solution or other physiologically acceptable solution, or each agent administered in separate parenteral dosage formulations. Where separate dosage formulations are used, the compositions can be administered at essentially the same time, i.e., concurrently, or at separately staggered times, i.e., sequentially and in any order; combination therapy is understood to include all these regimens.

Also included are patient care kits, comprising (a) a protein complex, as described herein; and optionally (b) at least one additional therapeutic agent (e.g., immunotherapy agent, chemotherapeutic agent, hormonal therapeutic agent, kinase inhibitor). In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a protein complex and optionally at least one additional therapeutic agent. For example, the kit includes a plurality of syringes, ampules, foil packets, or blister packs, each containing a single unit dose of a protein complex and optionally at least one additional therapeutic agent. The containers of the kits can be airtight, waterproof (e.g., impermeable to changes in moisture or evaporation), and/or light-tight.

The patient care kit optionally includes a device suitable for administration of the composition, e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

### Manufacturing and Purification Systems

Certain embodiments include methods and related compositions for manufacturing, expressing, and purifying a protein complex or protein component described herein. For example, certain embodiments relate to methods of manufacturing or otherwise preparing a pharmaceutical composition comprising a protein complex, by combining: (a) alpha-2-macroglobulin (A2M) proteins; and (b) serine protease proteins into a composition at a molar ratio [(a):(b)] of about 1:3 to about 1:1, thereby manufacturing the pharmaceutical composition comprising the protein complex. Specific embodiments include combining (a) and (b) into a composition at a molar ratio [(a):(b)] of about 1:3, 1:2.9, 1:2.8, 1:2.7, 1:2.6, 1:2.5, 1:2.4, 1:2.3, 1:2.1, 1:2, 1:1.9, 1:1.8, 1:1.7, 1:1.6, 1:1.5, 1:1.4, 1:1.3, 1:1.2, 1:1.1, or 1:1.

The A2M and/or serine protease proteins can be prepared or obtained by purifying from a biological sample or by recombinant techniques. For example, in some embodiments, the A2M and/or serine protease proteins are obtained by purifying the protein from the blood or plasma of a mammalian subject, for example, a human subject. Specific embodiments include obtaining and purifying the A2M proteins from plasma of a human subject prior to combining with serine protease proteins. Methods for purifying A2M from human plasma (plasma-enriched A2M; or A2M-PPP) are known in the art (see, for example, Jordan et al., Pain Physician, 23 (2): 229-23, 2020; U.S. Pat. No. 9,352,021).

Recombinant proteins can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, a recombinant protein may be prepared by a procedure including one or more of the steps of: (a) preparing a vector or construct comprising a polynucleotide sequence that encodes a protein described herein, which is operably linked to one or more regulatory elements; (b) introducing the vector or construct into a host cell; (c) culturing the host cell to express the protein; and (d) isolating the protein from the host cell.

To express a desired polypeptide, a nucleotide sequence encoding a protein may be inserted into appropriate expression vector(s), i.e., vector(s) which contain the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high-level expression of proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express recombinant proteins as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21 (DE3), a ADE3 lysogen of BL21 that supports T7-mediated expression and is deficient in Ion and ompT proteases for improved target protein stability. Also included are expression host strains carrying plasmids encoding (RNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS.TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif. 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326,681; and 7,029,872). Merely by way of example, recombinant yeast cultures can be grown in Fernbach Flasks or 15 L, 50 L, 100 L, and 200 L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., Proc. Natl. Acad. Sci. U.S.A. 91:3224-3227 (1994)). Also included are baculovirus expression systems, including those that utilize SF9, SF21, and T. ni cells (see, e.g., Murphy and Piwnica-Worms, Curr Protoc Protein Sci. Chapter 5: Unit 5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CVI line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for protein production, see, e.g., Yazaki and Wu, Methods in Molecular Biology, Vol. 248 (B. K. C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol, Biol. 55:121-131 (1995)).

Also included are high-throughput protein production systems, or micro-production systems. Certain aspects may utilize, for example, bexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009).

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with one or more polynucleotide sequences of interest may be cultured under conditions suitable for the expression and recovery of the protein from cell culture. Certain specific embodiments utilize serum free cell expression systems. Examples include HEK293 cells and CHO cells that can grow in serum free medium (see, e.g., Rosser et al., Protein Expr. Purif, 40:237-43, 2005; and U.S. Pat. No. 6,210,922).

A protein produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin, FLAG tags, poly-histidine tags (e.g., 6×His), cMyc tags, V5-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-performance liquid chromatography (HPLC) (e.g., Beckman and Waters HPLC). Exemplary chemistries for purification include ion exchange chromatography (e.g., Q. S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

Also included are methods of concentrating a protein or protein complex described herein, and compositions comprising the concentrated soluble protein or protein complex. In some aspects, such concentrated solutions of a protein or protein complex comprises proteins at a concentration of about or at least about 5 mg/mL, 8 mg/mL, 10 mg/mL, 15 mg/mL, 20 mg/mL, or more.

In some aspects, such compositions may be substantially monodisperse, meaning that a protein or protein complex exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, a protein or protein complex in a composition has a purity of at least about 90%, as measured according to routine techniques in the art. In certain embodiments, such as diagnostic compositions or certain pharmaceutical or therapeutic compositions, a protein or protein complex in a composition has a purity of at least about 95%, or at least about 97% or 98% or 99%. In some embodiments, such as when being used as reference or research reagents, a protein or protein complex can be of lesser purity, and may have a purity of at least about 50%, 60%, 70%, or 80%. Purity can be measured overall or in relation to selected components, such as other proteins, e.g., purity on a protein basis.

Purified proteins or protein complexes can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more biological activities can be measured according to in vitro or cell-based assays, which are optionally functionally coupled to a readout or indicator, such as a fluorescent or luminescent indicator of biological activity, as described herein.

In certain embodiments, as noted above, a composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, a protein or protein complex is made from a eukaryotic cell such as a mammalian or human cell in substantially serum free media. In certain embodiments, as noted herein, a composition has an endotoxin content of less than about 10 EU/mg of protein, or less than about 5 EU/mg of protein, less than about 3 EU/mg of protein, or less than about 1 EU/mg of protein.

In certain embodiments, a composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15:1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade proteins or protein complexes can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of a protein or protein complex and variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. A protein or protein complex with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains a protein or protein complex of the present disclosure.

Although the foregoing embodiments have been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Characteristics of A2M: PPE Protein Complexes

Experiments were performed to test the characteristics of protein complexes composed of different molar ratios of A2M and the PPE protein ‘Mutant F’ (MutF, SEQ ID NO: 5).

ALAT Protection Assay. MutF (400 nM) was mixed with different concentration of A2M to reach various molar ratios, followed by 30 minutes incubation at 37° C. A protection assay was performed in 384-black well coated plate by adding 10 μL of the sample with either 10 μL of PBS or 10 μL of ALAT 2 μM or at different concentrations in duplicates. 20 μL of the substrate AAPV-AMC at a concentration of 100 μM was added. Kinetics were measured on Varioskan LUX: 380 nm ex/460 nm em (5 nm bandwidth), 37° C., top, 100 msec, 2 min for 16 reads. Vo was calculated as activity measurement.

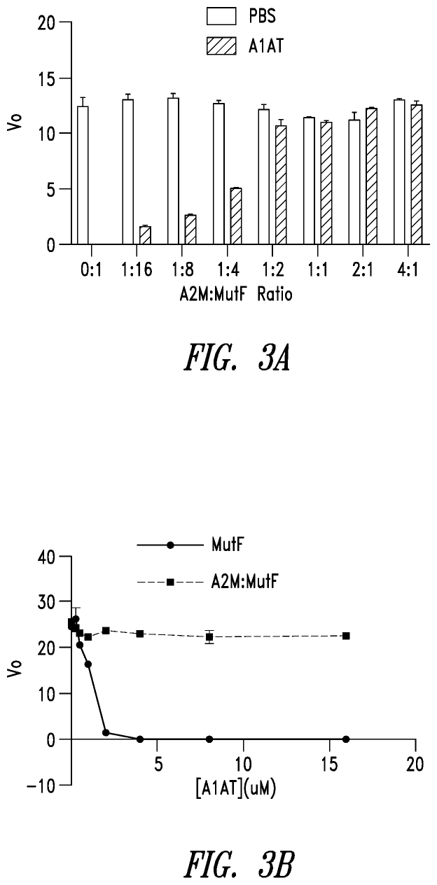

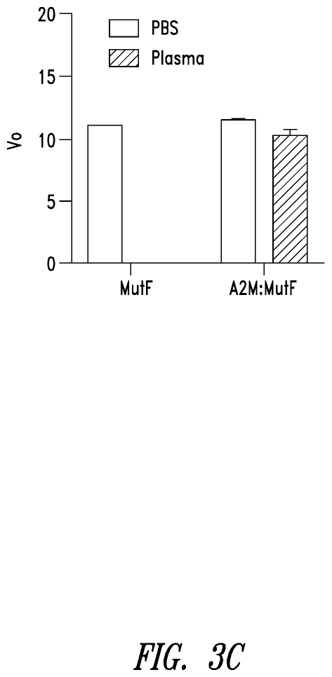

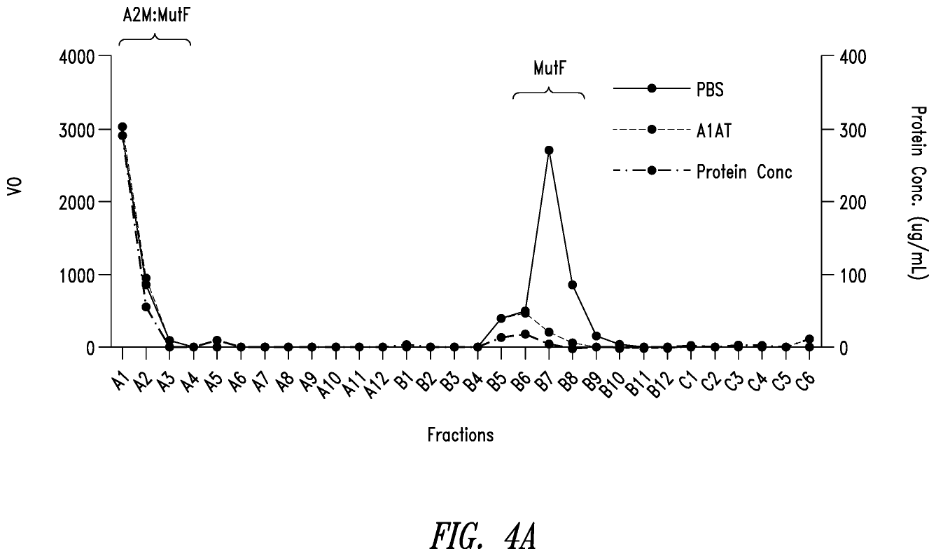

As shown in FIGS. 3A-3B, A2M:MutF protein complexes at 1:2 molar ratio provides the best protection against A1AT (3A), and increasing concentration of ALAT doesn't disturb the protection of MutF by A2M (3B).

Plasma Protection Assay. MutF (400 nM) was mixed with 200 nM A2M to reach a 1:2 molar ratio (A2M:MutF), followed by 30 minutes incubation at 37° C. in PBS. A plasma protection assay was performed in 384-black well-coated plate by adding 10 μL of sample with either 10 μL of PBS or 10 μL of mouse plasma samples in duplicates. 20 μL of the substrate AAPV-AMC at a concentration of 100 μM was added. Kinetics were measured on Varioskan LUX: 380 nm ex/460 nm (5 nm bandwidth), 37° C., top, 100 msec, 2 min for 16 reads. Vo was calculated as activity measurement.

As shown in FIG. 3C, plasma inhibits MutF activity on its own but does not inhibit its activity when complexed with A2M:MutF at a 1:2 molar ratio.

Purification of N17350-A2M by column chromatography. MutF was mixed with A2M to reach a 1:2 molar ratio (A2M:MutF), followed by a 30 minutes incubation at 37° C. AKTA pure chromatography system was used for protein purification. For the cation exchange column, the PD10 desalting column was used to buffer exchange the protein mix into 50 mM sodium acetate, pH5.0 solution. Samples were loaded on pre-washed/equilibrium HiTrap SP column with 0.5M NaCl, 50 mM sodium acetate, pH 5.0 elute in 20 fractions (1 mL/fraction). For the size exclusion column, PD10 desalting column was used to buffer exchange the protein mix into 50 mM sodium phosphate, 150 mM NaCl, pH7. Samples were loaded on pre-washed Superose 6 10/300 increase. Flow rate were set as 0.5 mL/min to collect 1 mL per fraction. Each fraction was diluted 1:250 in PBS for A2M:MutF A1AT protection assays. Protein concentration was measured using Pierce™ BCA protein assay kit (ThermoFisher Scientific, see Manufacturer's instruction). For the ALAT protection assay, 10 μL of each fraction dilution were coated in 384-black well plate with either 10 μL of PBS or 10 μL of A1AT 2 μM or at different concentrations in duplicates. 20 μL of the substrate AAPV-AMC at a concentration of 100 μM was added. Kinetics were measured on Varioskan LUX: 380 nm ex/460 nm em (5 nm bandwidth), 37° C., top, 100 msec, 2 min for 16 reads. Vo was calculated as activity measurement.

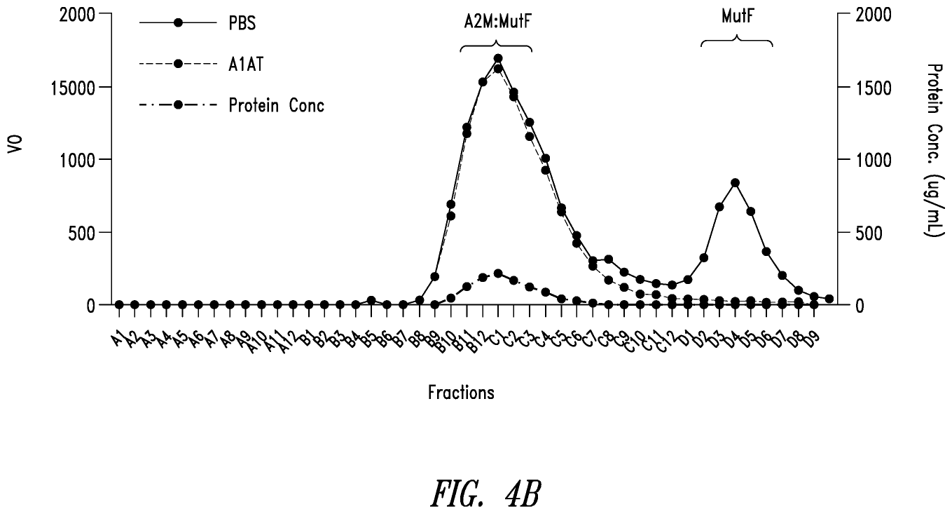

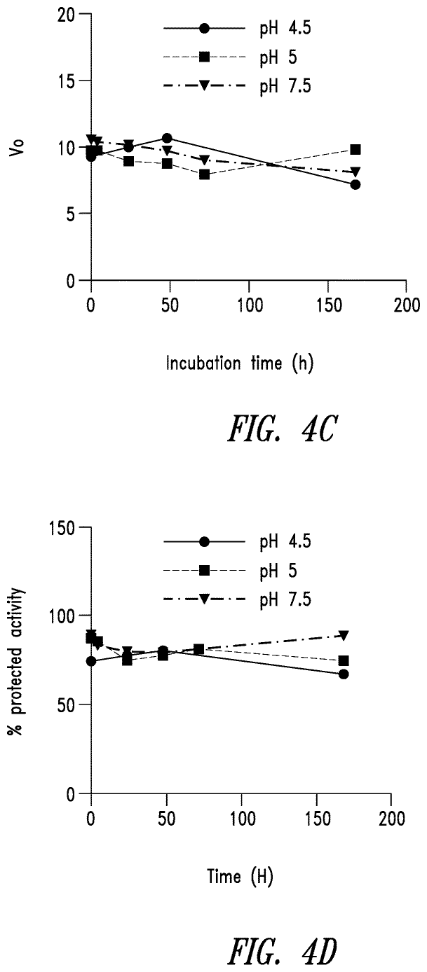

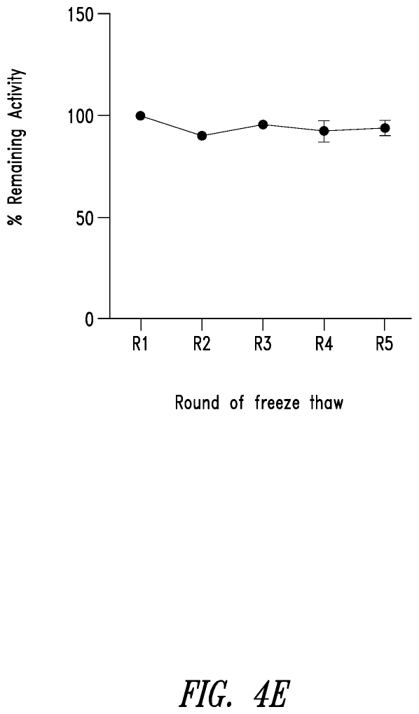

The results in FIGS. 4A-4E show that the A2M:MutF protein complex is stable. FIG. 4A shows MutF activity and concentration of the different fractions of A2M:MutF complex after cation exchange column separation in the presence or absence of A1AT. FIG. 4B shows MutF activity and concentration of the different fractions of A2M:MutF complex after size exclusion column separation in the presence or absence of A1AT. FIGS. 4C-4D show that A2M:MutF complexes are stable across a broad pH range as measured by enzyme activity (4C) and ALAT protection (4D). FIG. 4E shows that A2M:MutF complexes are stable over multiple freeze/thaw cycles, as measured by enzyme activity.

Activity & ALAT protection in cell lysates. Tumor cells were trypsinized, washed with PBS, counted, and diluted in serum free media. 40K of tumor cells were added to a V bottom plate, followed by adding 40 μL of different conditions (SFM, A2M, 400 nM N17350, or pre-mixed A2M:MutF (800 nM-400 nM). The plate was incubated at 37° C. for 30 minutes. After incubation the plate was centrifuged at 300×g for 5 minutes and washed with an additional 200 μL of PBS per well. 25 μL of cytoplasmic lysis buffer was added to each well (10 mM HEPES pH8, 10 mM KCl, 0.1 mM EDTA, 0.3% NP-40), and incubated on ice for 20 minutes with 10 seconds vortex every 5 minutes. The plate was centrifuged at 300×g for 5 minutes. The supernatant was carefully transferred to a new 96-well plate for A2M:MutF activity and protection assay measurement. Here, 10 uL of the supernatant was coated in 384-black well plates with either 10 μL of PBS or 10 μL of A1AT 2 μM or at different concentrations in duplicates. 20 μL of the substrate AAPV-AMC at a concentration of 100 μM was added. Kinetics were measured on Varioskan LUX: 380 nm ex/460 nm em (5 nm bandwidth), 37° C., top, 100 msec, 2 min for 16 reads. Vo was calculated as activity measurement.

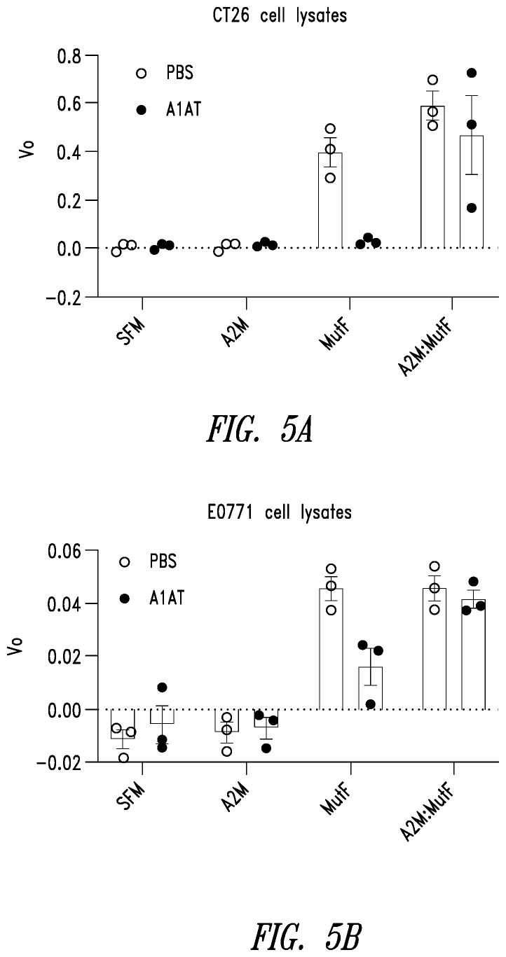

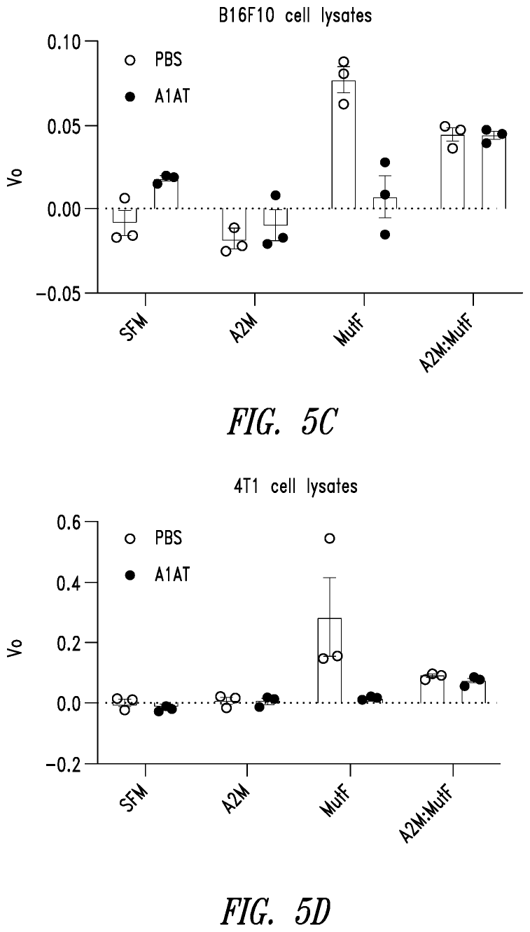

The results in FIGS. 5A-5D show that A2M:MutF protein complexes can enter the cells and remain in the protein complex form intracellularly since ALAT is not able to inhibit its activity.

CD95-C cleavage assay. MutF was mixed with different concentrations of A2M to reach various molar ratios, followed by a 30-minute incubation at 37° C. in PBS. Recombinant CD95-C terminal protein (Wuxi) was incubated with pre-mixed A2M:MutF protein complexes at different molar ratios for 1 hr at 37° C. Samples containing 1 ug of CD95-C were loaded on 17.5% SDS-PAGE gels, and stained with One-step Blue (Biotium). Gels were imaged by iBright 1500.

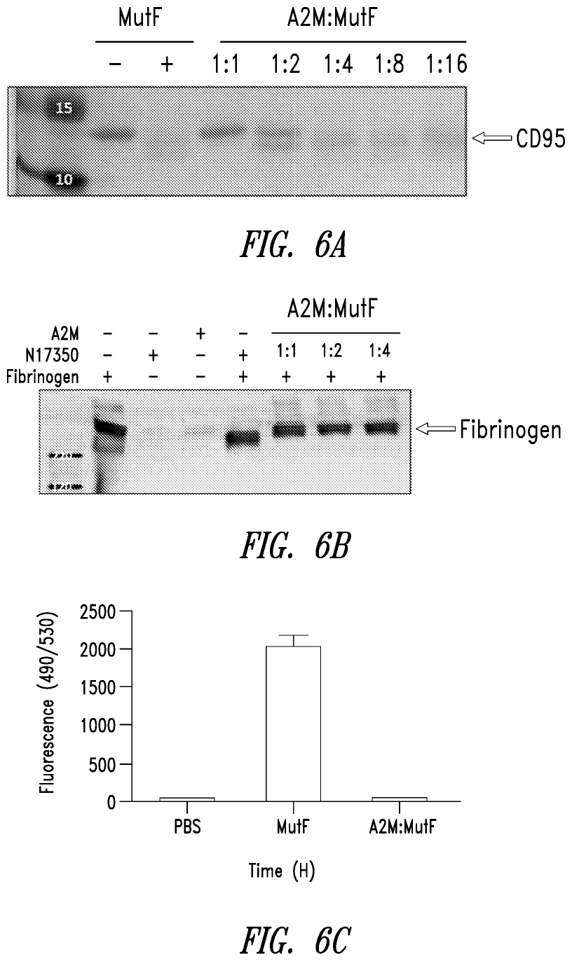

As shown in FIG. 6A, A2M:MutF protein complexes ranging from a 1:2 to 1:16 molar ratio cleave CD95 as efficiently as MutF alone.

Fibrinogen cleavage assay. MutF was mixed with different concentrations of A2M to reach various molar ratios, followed by a 30-minute incubation at 37° C. in PBS. Human fibrinogen (Sigma) was incubated with pre-mixed A2M:MutF protein complexes at different molar ratios for 1 hr at 37° C. Samples containing 5 ug of fibrinogen were loaded on 4-15% gel and fibrinogen was detected by Western Blot with an anti-fibrinogen (cell signaling Technology). Gels were imaged by iBright 1500.

As shown in FIG. 6B, the A2M:MutF protein complexes do not cleave fibrinogen, as compared to MutF on its own, which does cleave fibrinogen.

Elastin cleavage assay. MutF was mixed with different concentrations of A2M to reach various molar ratios, followed by a 30-minute incubation at 37° C. in PBS. Elastin-F (1:100) was incubated with pre-mixed A2M:MutF protein complexes overnight at 37° C., and the degraded fluorescent signal was measured in the supernatant.

As shown in FIG. 6C, the A2M:MutF protein complexes do not cleave elastin, as compared to MutF on its own, which does cleave elastin.

Coagulation Assay. Mice were injected with 200 μL of 480 ug of MutF or purified A2M:MutF protein complexes at a 1:2 molar ratio. Blood was collected in sodium citrate tubes and centrifuged at 1500×g for 15 minutes. 300 μl of plasma were frozen and shipped to IDEXX to be analyzed for Prothrombin time, Partial Thromboplastin time, and concentration of Fibrinogen.

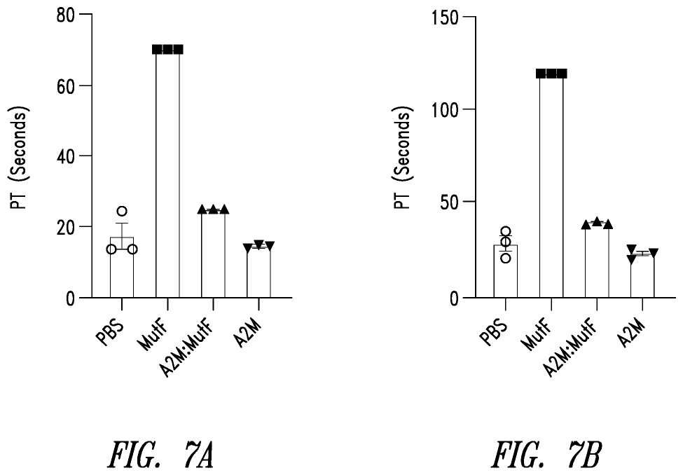

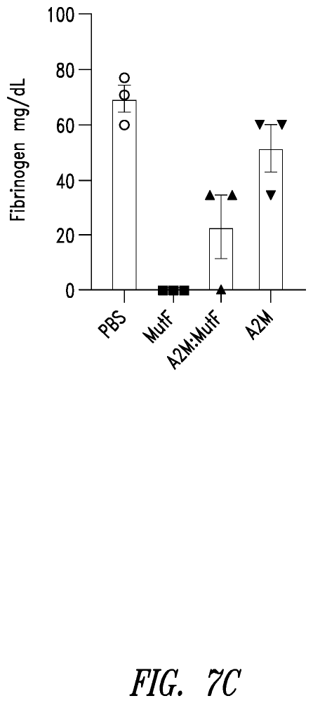

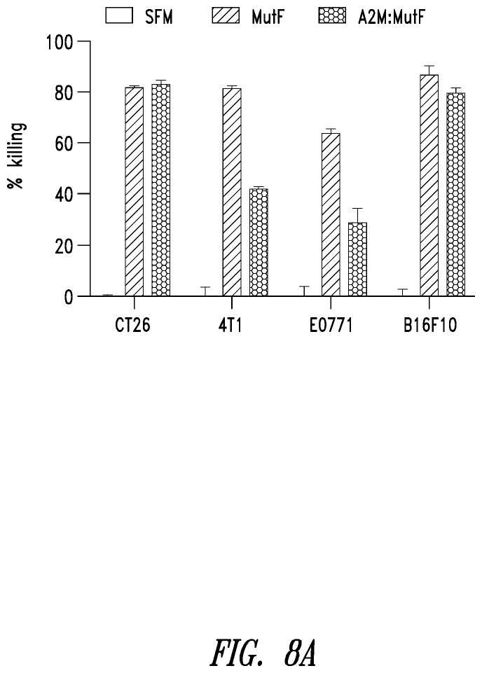

The results in FIGS. 7A-7C show that A2M:MutF protein complexes do not induce coagulation effects, as compared to MutF on its own, which in contrast increases PT and PTT times and causes reductions in fibrinogen levels.

Cell-Killing assay. About 20K-40K of cancer cells were plated in a black-well coated 96 well plates overnight to settle down. On the second day, each well was washed with 200 μL of serum free media once, followed by different treatments in triplicates. 24 h after treatment, cells were incubated with pre-diluted Calcein-AM solution (ThermoFisher, 4 μM in HBSS with Ca2+Mg2+) for 45 minutes. Calcein AM was dumped and 100 μL of HBSS with Ca2+Mg2+ was added to each well and fluorescence was measured by Varioskan LUX at 485/520 nm (20 bandwidth).

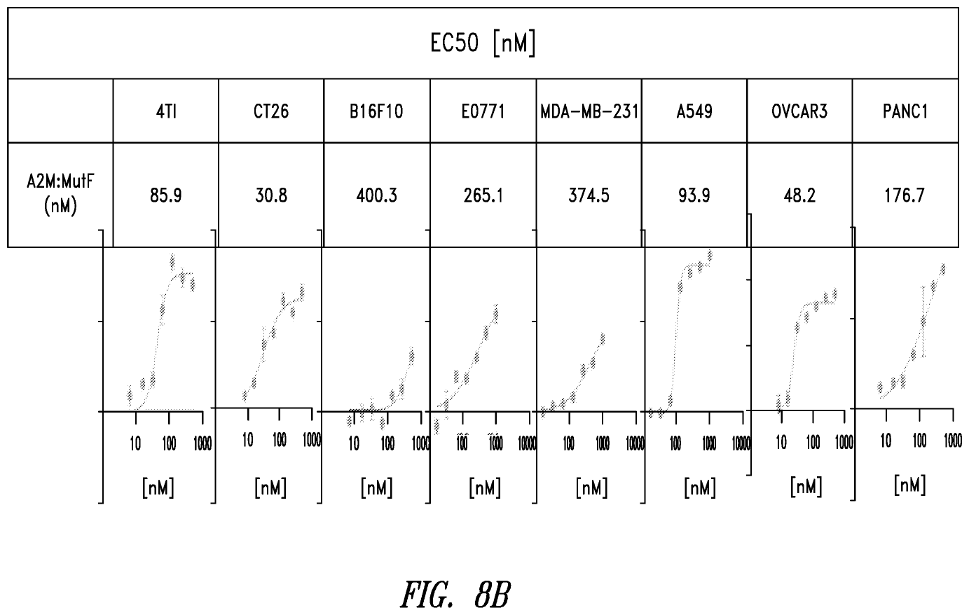

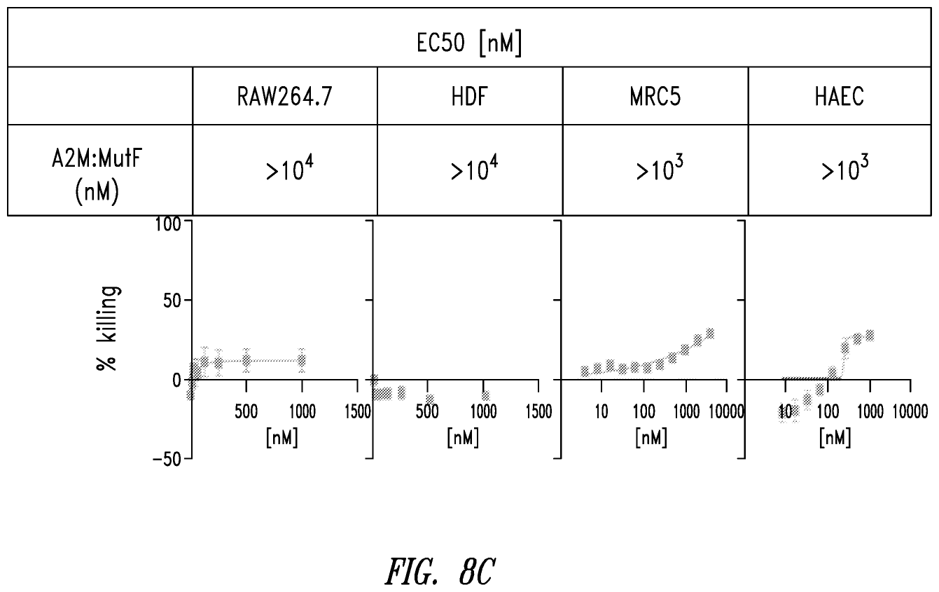

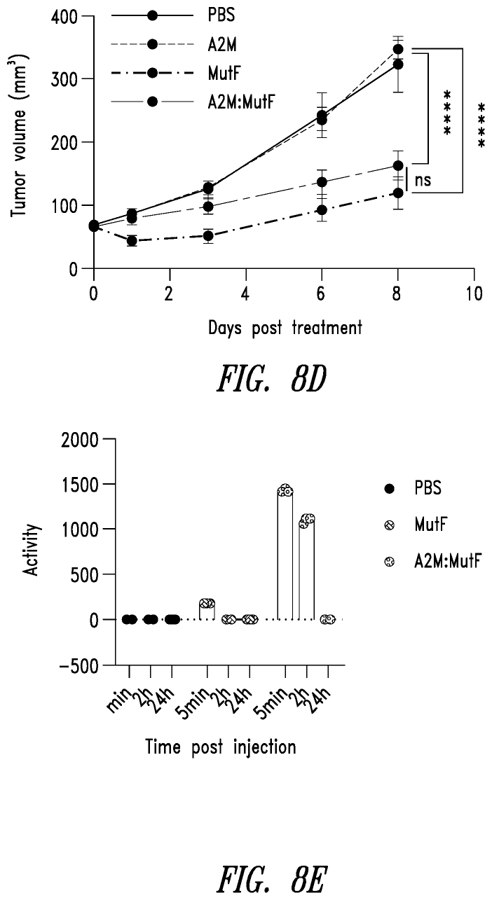

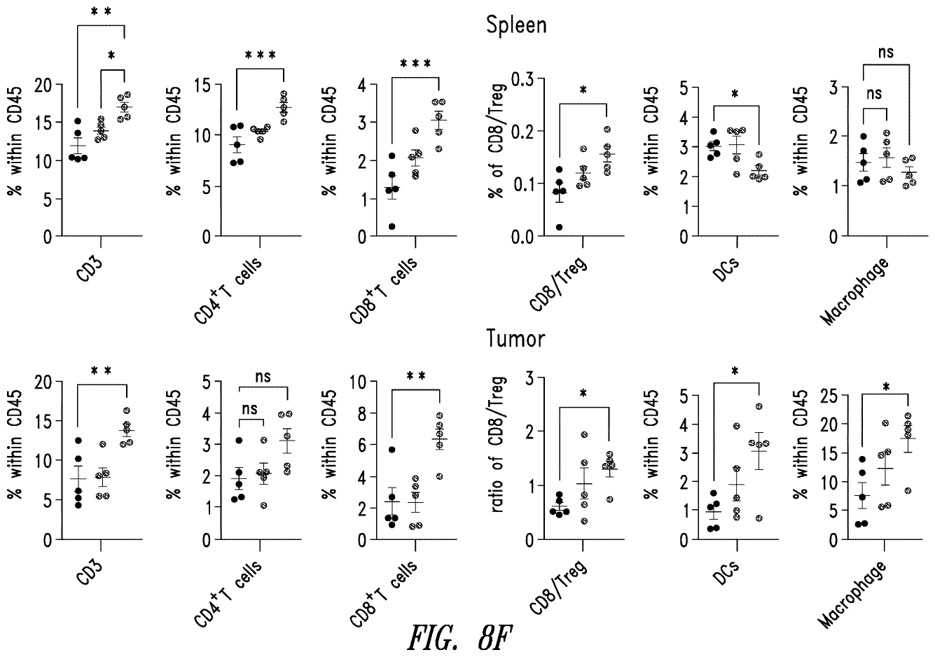

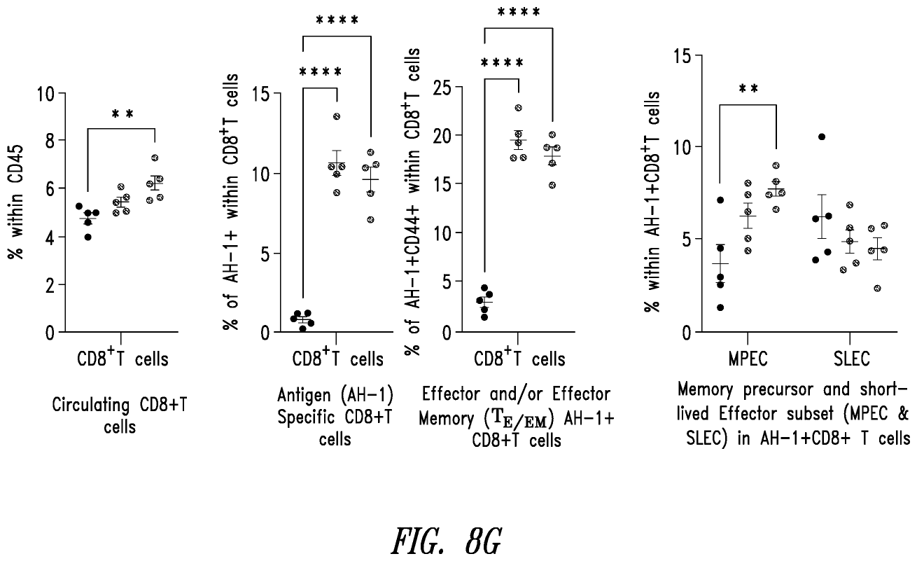

As shown in FIG. 8A, A2M:MutF protein complexes at a 1:2 molar ratio induce killing of a variety of cancer cells in vitro, comparable in most instances to the activity of MutF on its own. FIG. 8B shows broad cytotoxicity of A2M:MutF (1:2) protein complex towards cancer cells of different anatomical origin, and FIG. 8C shows that the complex does not kill non-cancer cells.

In vivo efficacy. About one million CT26 cells were implanted in the flank of 7-8 weeks-old BALB/c mice. Once the tumors reached ~80 mm³, tumors were treated with 100 ug of MutF, 100 ug of A2M:MutF (1:2), or 1.4 mg of A2M (corresponding to the amount to generate A2M:MutF molar ratio at 1:2). The tumors were monitored every two days.

As shown in FIG. 8D, A2M:MutF protein complexes at a 1:2 molar ratio induce killing of cancer cells in vivo, comparable to the cell-killing activity of MutF on its own. FIG. 8E shows that A2M:MutF protein complex has an improved functional PK profile (enzymatic activity in plasma) relative to MutF alone following intravenous administration. FIG. 8F shows that A2M:MutF protein complex induces a favorable immune profile in the CT26 model (left to right in each graph is PBS, MutF, A2M:MutF). FIG. 8G shows that A2M:MutF protein complex elicits a tumor antigen-specific CD8+ T cell response in the CT26 model (left to right in each graph is PBS, MutF, A2M:MutF). Induction of effector & memory T cells is indicative of a functional adaptive immune response.

Evaluating selective cancer cell-killing in ovarian cancer patient samples. Cancer and non-cancer cells were isolated from primary tumors, intraperitoneal (IP) fluid, omental adipose tissue (common metastatic site), and blood from 6 ovarian cancer patients. The following cells were isolated and tested: cancer cells (digested tumor with depletion of fibroblasts, CD45+ cells, and EpCAM+/high cells), non-cancer immune cells (digested omental cells with depletion of neutrophils or CD45+ cells from tumor tissue), peripheral blood mononuclear cells (PBMCs; B cells, T cells, monocytes, and NK cells), fibroblasts (isolated from tumor samples using fibroblast isolation kit), and IP cells (composed of >90% CD45+ immune cells, including B, T, myeloid, NK cells). Isolated cells were plated and exposed to A2M:MutF, doxorubicin (standard of care chemotherapy), or oxaliplatin (standard of care chemotherapy) for 24 hours. Cell viability was assessed by Calcein-AM.

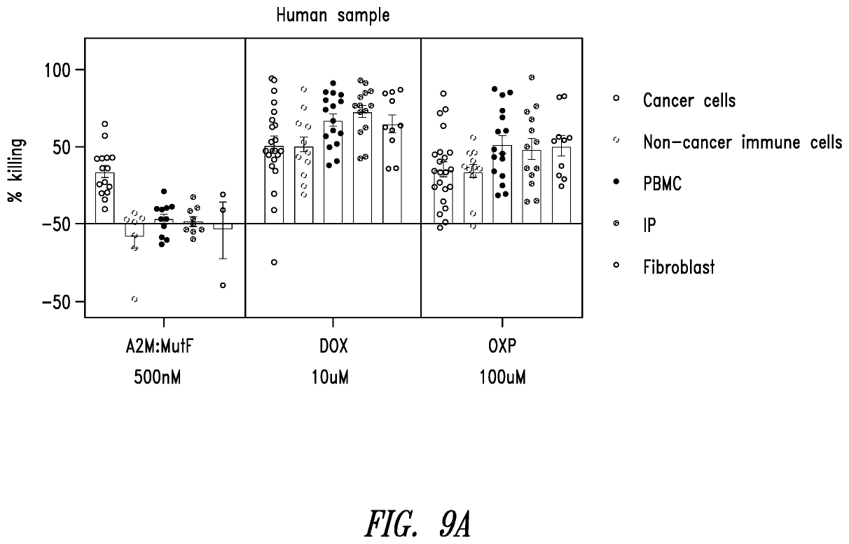

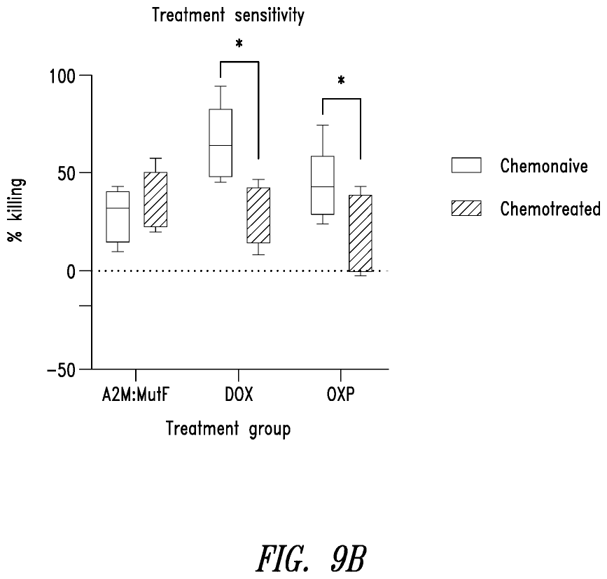

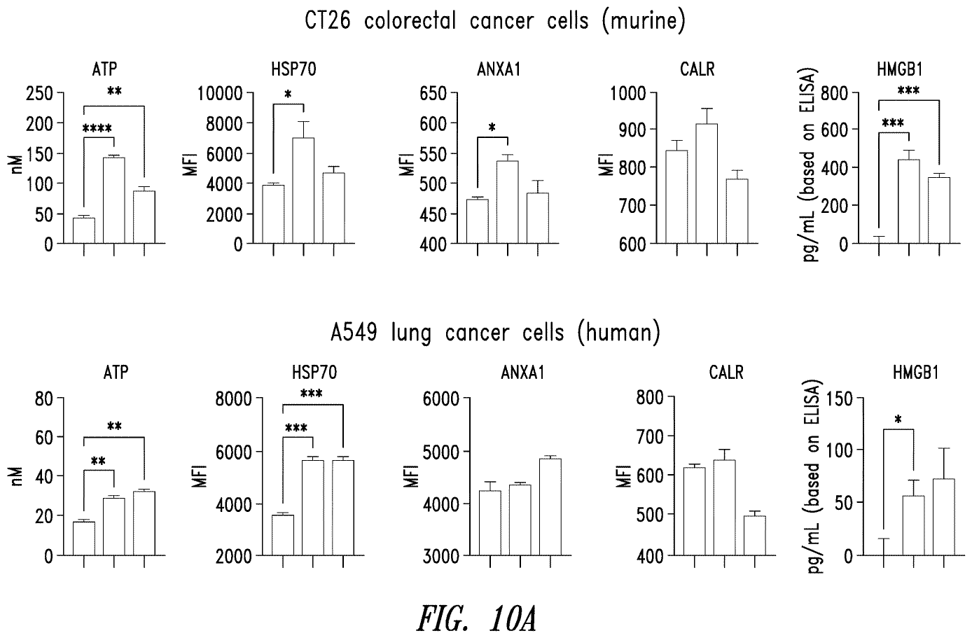

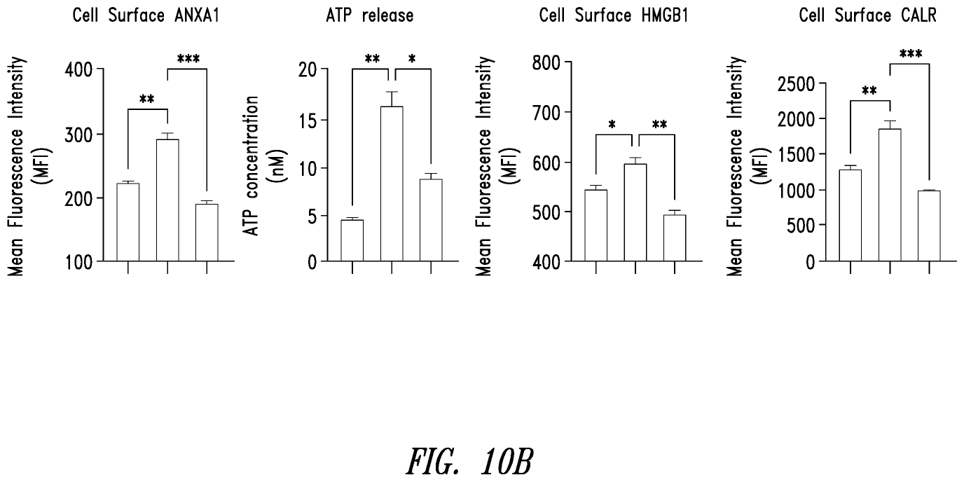

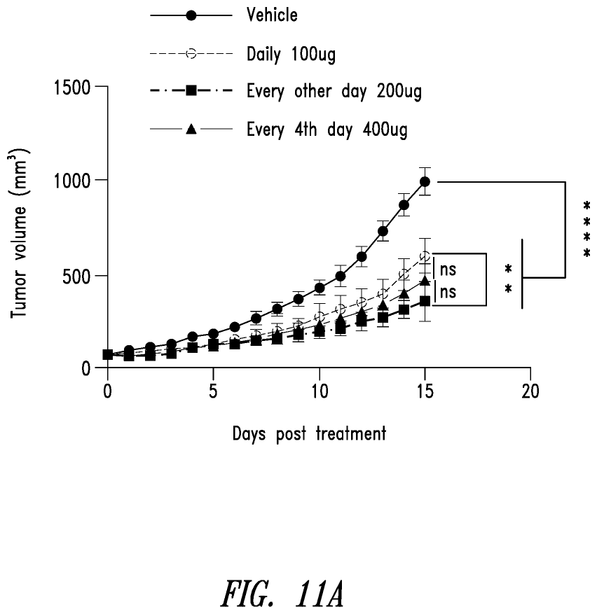

FIG. 9A shows that A2M:MutF protein complex, in contrast to doxorubicin and oxaliplatin, has a wide therapeutic window as shown by killing human ovarian cancer cells without killing non-cancer cells from patients. FIG. 9B shows that A2M:MutF comparably kills cancer cells isolated from chemo-naïve and chemo-treated patients, in contrast to doxorubicin and oxaliplatin, which show reduced killing of cancer cells isolated from chemo-treated relative to chemo-naïve patients.

Evaluating immunogenic cell death (ICD). CT26 (murine colon), A549 (human lung), and ovarian patient cancer cells were treated with A2M:MutF and oxaliplatin (known ICD inducer in certain cell types at high concentrations) for 24 hours. Immunogenic cell markers were evaluated, including HSP70, ATP release, HMGB1, and CALR (see, for example, Fucikova et al., Cell Death Dis. 11 (11): 1013, 2020).

FIG. 10A shows that A2M:MutF protein complex induces ICD markers in CT26 and A549 cells. FIG. 10B shows that A2M:MutF protein complex induces ICD markers in human ovarian patient-derived tumor cells (left to right in each graph are CTRL, A2M:MutF, oxaliplatin).

Evaluating the dose and schedule of A2M:MutF. CT26 colon cancer cells were injected into the flanks of mice and grown until they reached about 80 mm³. Multiple intravenous doses of A2M:MutF protein complex or vehicle were administered as below:
Vehicle: Day 0, 2, 4, and 6, and Day 8, 10, 12 and 14;
A2M:MutF 100 μg: Every single day for 2 weeks (daily);
A2M:MutF 200 μg: Day 0, 2, 4, 6 and Day 8, 10, 12 and 14 (every other day); and
A2M:MutF 400 μg: Day 0, 4, 8, and 12 (every 4th day).

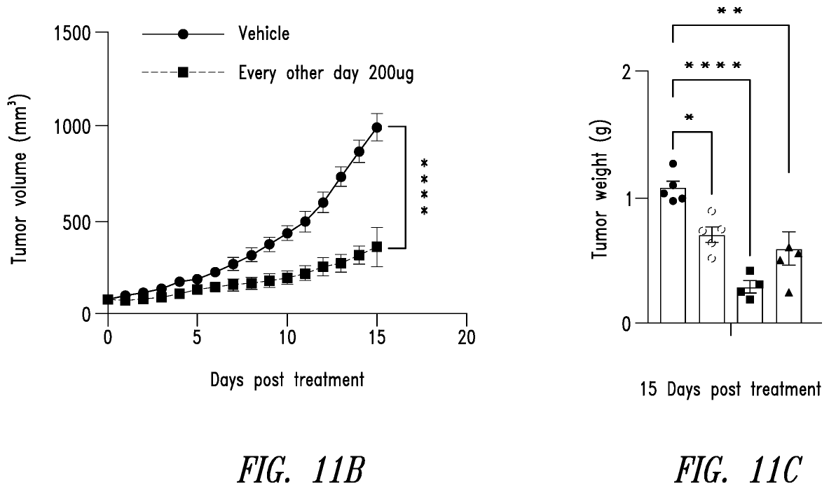

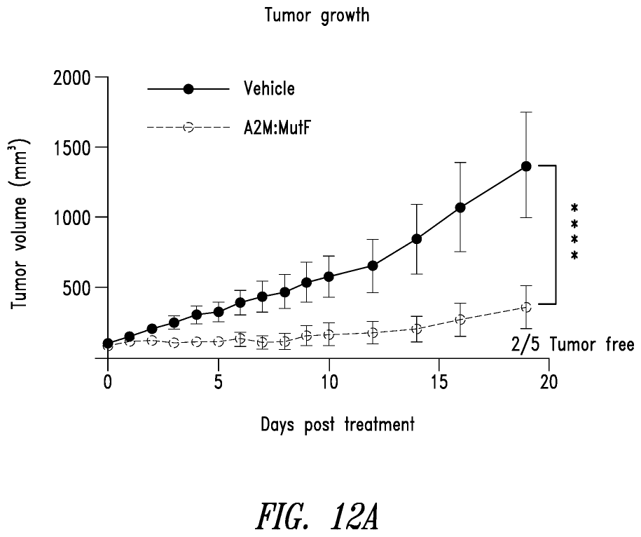

Tumor growth and weight were assessed. FIGS. 11A-11B show tumor growth post-treatment, and FIG. 11C shows tumor weight at 15 days post-treatment (11C from left to right: vehicle every other day, A2M:MutF 100 μg daily, A2M:MutF 200 μg every other day, A2M:MutF 400 μg every 4^{th} day).

Evaluating anti-tumor efficacy in syngeneic mice tumor models. CT26 (highly immunogenic model of colon), MC38 (warm immunogenic model of colon), and B16F10 (cold immunogenic model of skin cancer and lung metastasis) cancer cells were injected into the flanks of syngeneic B-cell deficient Jh-BALB/c or C57BL/6 mice and grown until they reached about 80-100 mm³. A2M:MutF protein complex (400 μg) or vehicle were injected intravenously every other day for 2-3 weeks. Tumor growth was monitored.

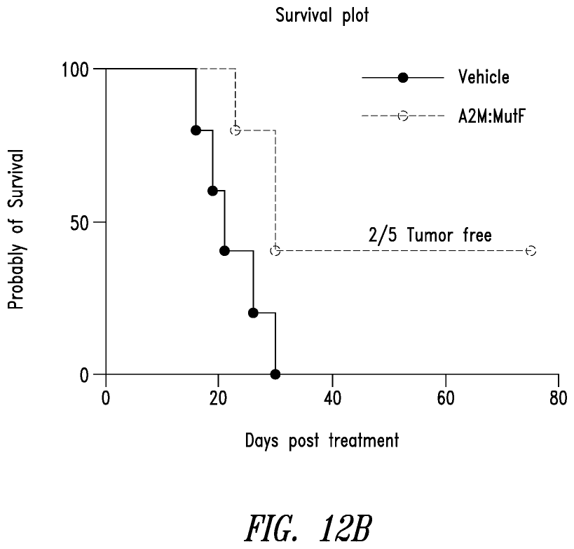

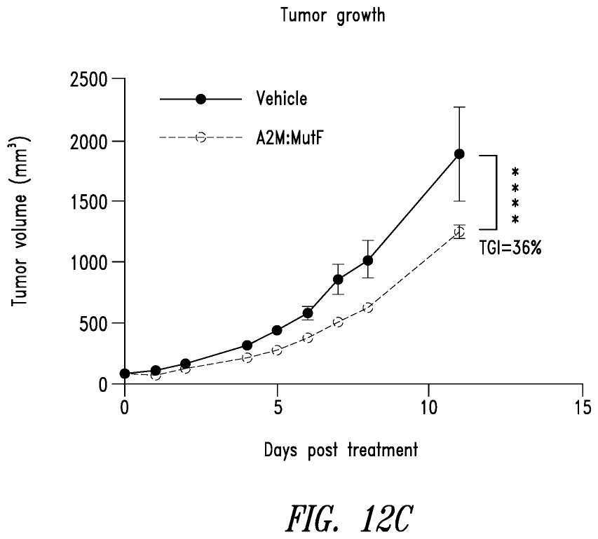

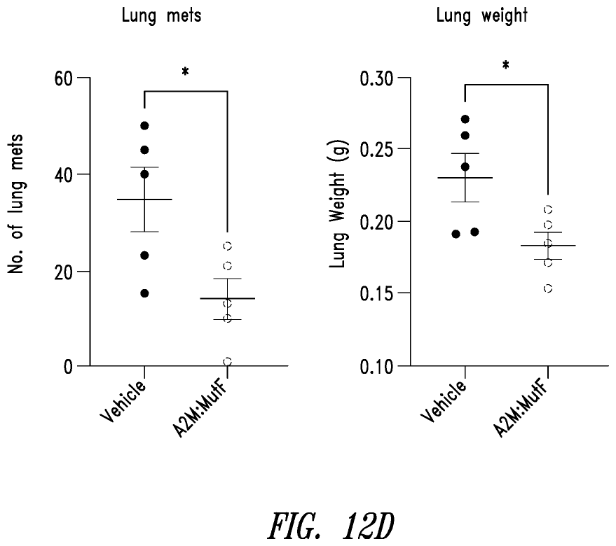

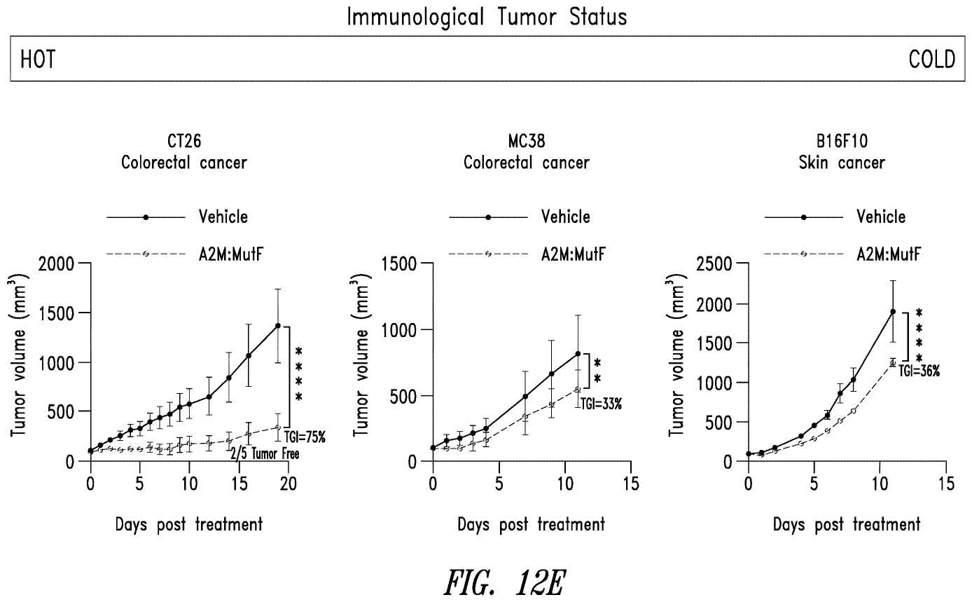

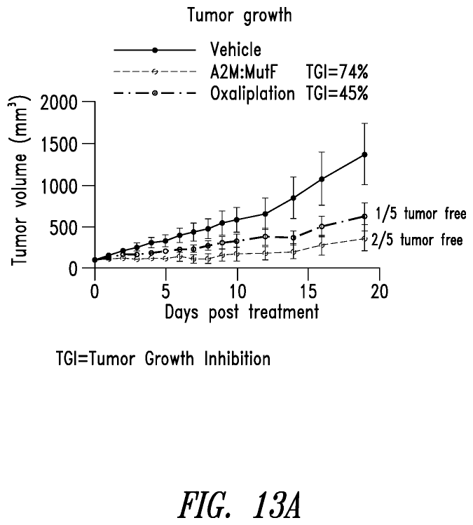

FIGS. 12A-12B show that A2M:MutF protein complex effectively attenuates tumor growth in Jh-BALB/c CT26 colorectal cancer model. FIGS. 12C-12D show that A2M:MutF protein complex treats primary tumor and metastasis in a Jh-C57BL/6 B16F10 melanoma model. FIG. 12E shows that A2M:MutF protein complex displays efficacy across a range of tumors with variable immunological status.

Evaluating anti-tumor efficacy relative to SoC chemotherapy. CT26 colon cancer cells were injected into the flanks of syngeneic B-cell deficient Jh-BALB/c mice and grown until they reached about 80-100 mm³. A2M:MutF protein complex (400 μg every other day for 3 weeks) or oxaliplatin (6 mg/kg on day 0 and 2) were injected intravenously. Tumor growth and overall survival were monitored.

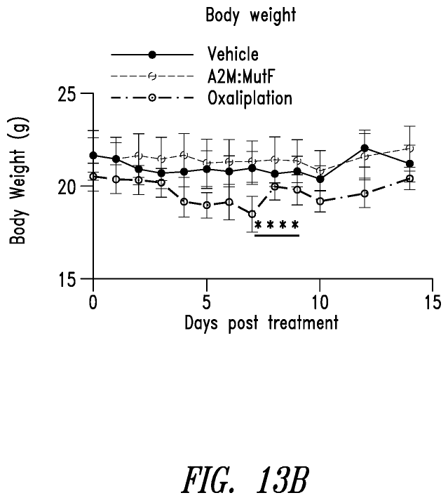

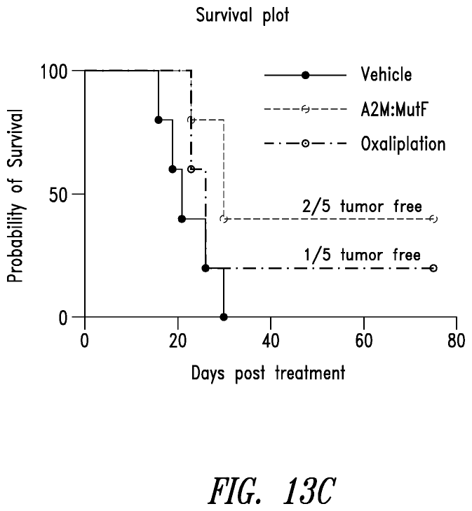

FIGS. 13A-13C show that A2M:MutF protein complex has improved anti-tumor efficacy relative to SoC chemotherapy (oxaliplatin) without observed toxicity.

Evaluating anti-tumor efficacy towards human cancer cells in xenograft models (NU/NU and NCG female mice). HCT116 (human colorectal cancer), HT29 (human colorectal cancer), PC3 (human prostate cancer), NCI-H358 (human lung cancer), A549 (human lung cancer), ovarian patient derived (CDX) model, and breast cancer (PDX) model cancer cells were injected into the flanks of mice and grown until they reached about 80-100 mm³. A2M:MutF protein complex (400 μg) or vehicle were injected intravenously every other day for 2-3 weeks. Tumor growth and overall survival were monitored.

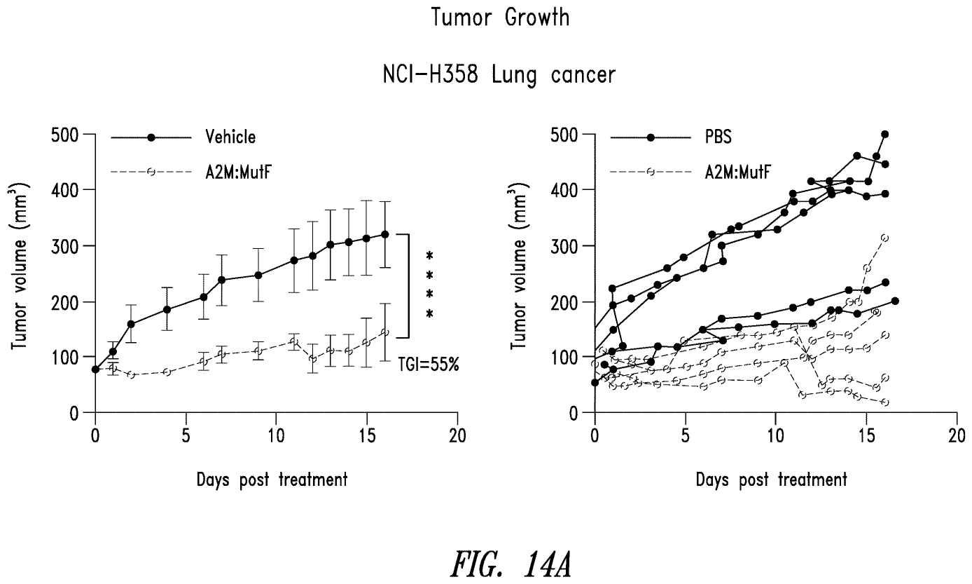

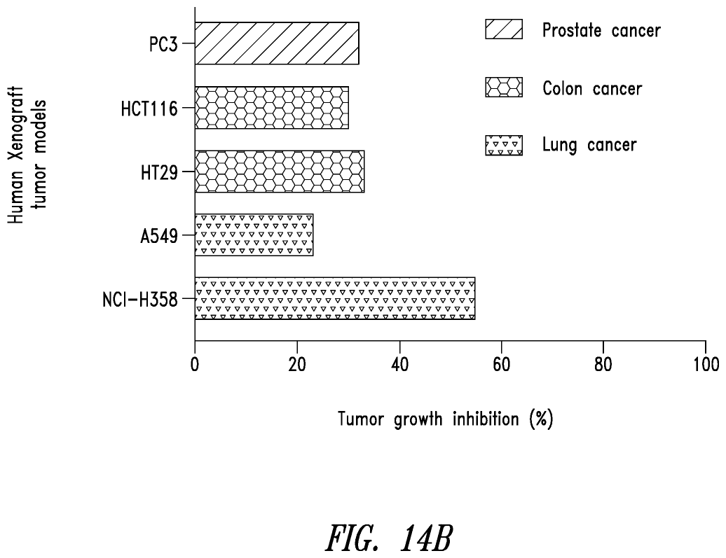

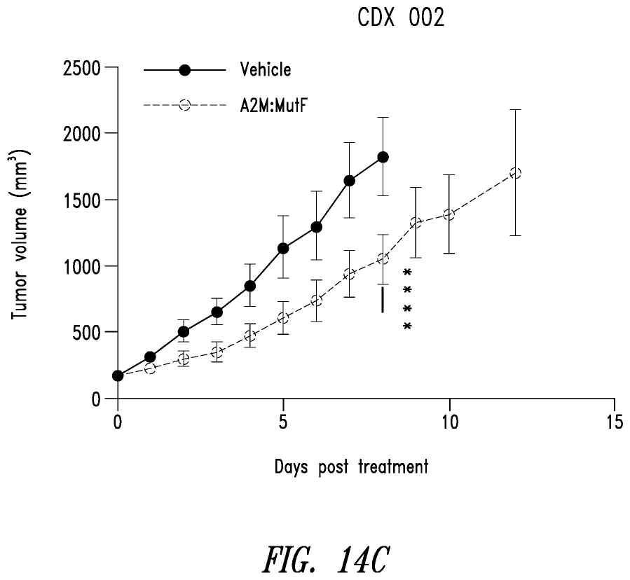

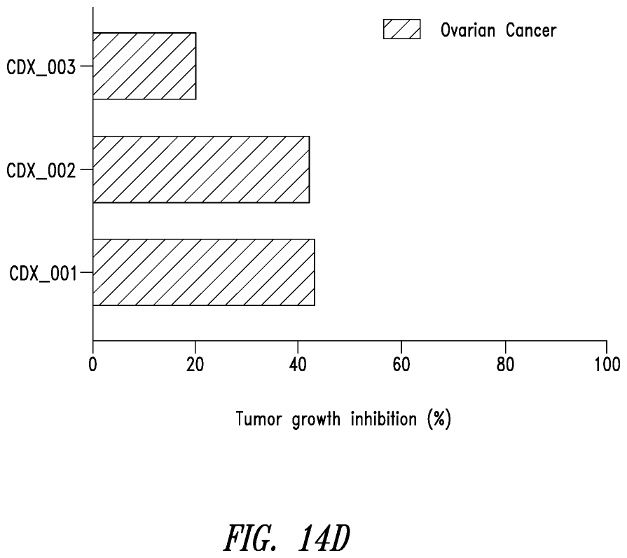

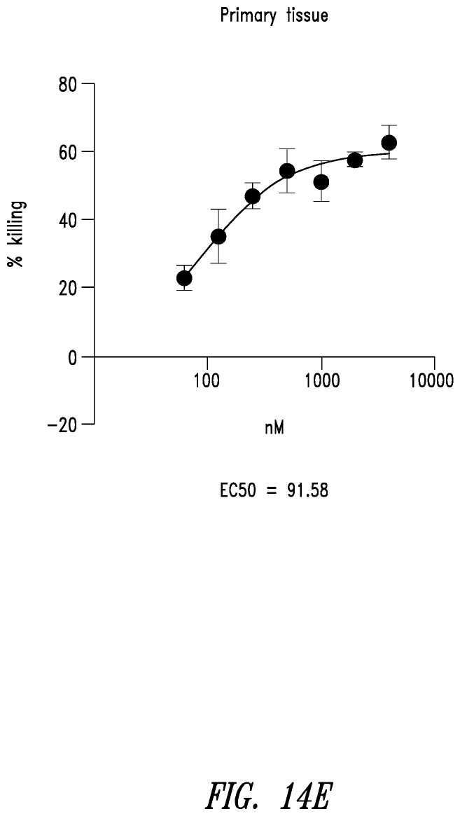

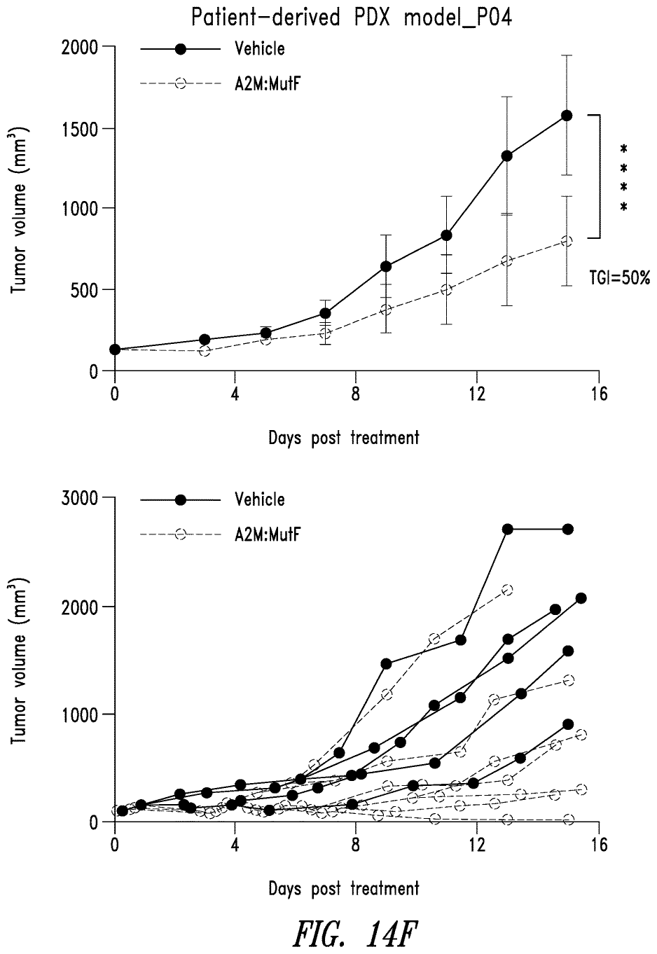

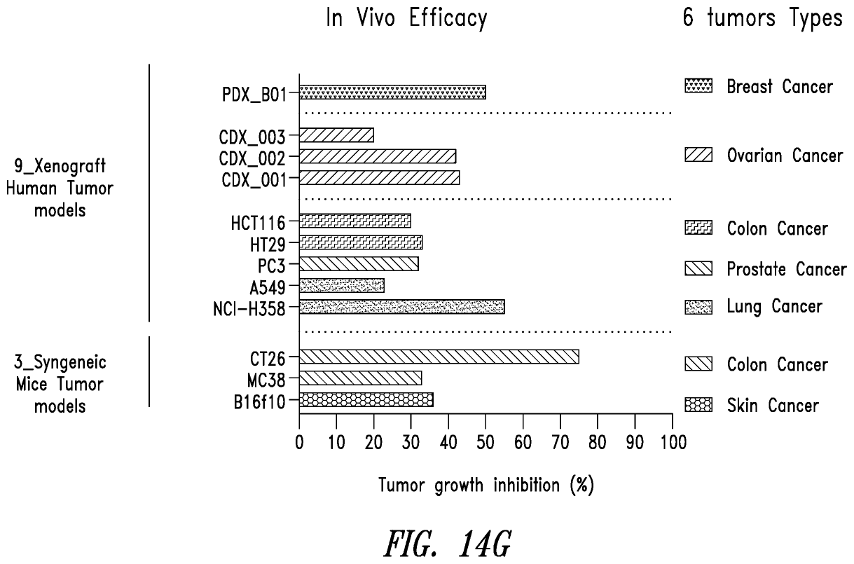

FIG. 14A shows the efficacy of A2M:MutF protein complex in a lung cancer human xenograft model. FIG. 14B summarizes the efficacy of A2M:MutF protein complex across a variety of prostate cancer, colon cancer, and lung cancer models. FIG. 14C shows that A2M:MutF protein complex effectively kills human ovarian patient-derived tumor cells (from patient CDX_002) in a xenograft mouse model, and FIG. 14D summarizes the efficacy of A2M:MutF protein complex in this model across three ovarian cancer patients (patient histories: diagnoses, Grade 3; treatment, CDX_001 and CDX_003 chemo-naïve and CDX_002 treated with 3 cycles of carboplatin+paclitaxel+keytruda). FIGS. 14E-14F show that A2M:MutF protein complex effectively kills patient-derived breast cancer cells in vitro and in vivo (patient history: diagnosis, breast cancer HER2− and ER+; treatment, chemo-naïve). FIG. 14G summarizes the in vivo efficacy of A2M:MutF protein complex across a variety of human tumors and shows that the efficacy is independent of tumor genetics or immune status.

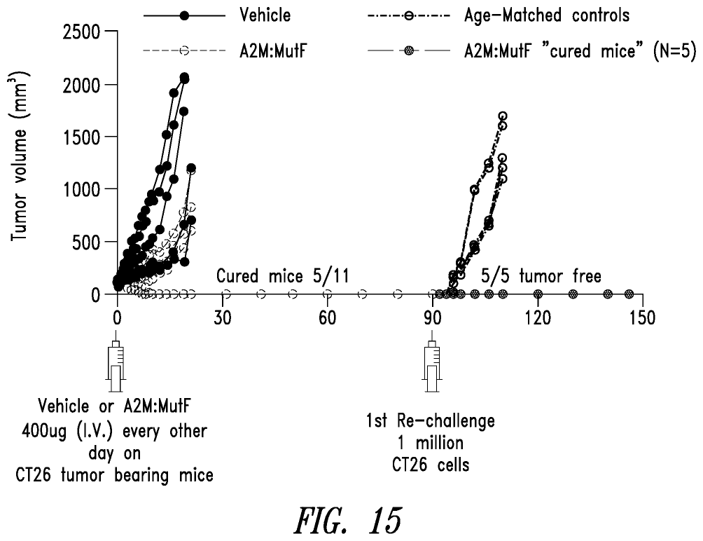

FIG. 15 shows that mice treated with A2M:MutF protein complexes are tumor-free (5/11) after initial challenge with CT26 colorectal cancer cells, and that all of these mice (5/5) retain their tumor-free status upon re-challenge with CD26 cells. This study suggests that treatment with A2M:MutF protein complexes induces a tumor-specific immune memory response.

1. A pharmaceutical composition, comprising a protein complex of:
(a) alpha-2-macroglobulin (A2M) proteins; and
(b) serine protease proteins,
wherein (a) and (b) are present in the composition at a molar ratio [(a):(b)] of about 1:3 to about 1:1.

2. The pharmaceutical composition of claim 1, wherein the A2M proteins of (a) and the serine protease proteins of (b) are bound together in the protein complex, and optionally wherein the protein complex:
(i) retains CD95 (Fas Receptor) protease cleavage activity and cancer cell-killing activity of (b);
(ii) sterically hinders binding of (b) to fibrinogen and reduces or inhibits fibrinogen cleavage activity of (b); and
(iii) sterically hinders binding of (b) to serine protease inhibitors (including alpha-1 antitrypsin (A1AT)).

3. The pharmaceutical composition of claim 1 or 2, wherein (a) comprises, consists, or consists essentially of an amino acid sequence that is at least 80, 85, 90, 95, 98, 99, or 100% identical to a sequence selected from Table A1, or a functional fragment thereof.

4. The pharmaceutical composition of claim 3, wherein the functional fragment thereof comprises, consists, or consists essentially of about 100, 200, 300, 400, 500, 600, 700, 800, 900, 1000, 1200, 1300, or 1400 contiguous amino acids of a sequence selected from Table A1.

5. The pharmaceutical composition of claim 4, wherein the functional fragment thereof is composed of approximately residues 1-1400, 1-1300, 1-1200, 1-1100, 1-1000, 1-900, 1-800, 1-700, 1-600, 1-500, 1-400, 1-300, 1-200, 100-1400, 100-1300, 100-1200, 100-1100, 100-1000, 100-900, 100-800, 100-700, 100-600, 100-500, 100-400, 100-300, 100-200, 200-1400, 200-1300, 200-1200, 200-1100, 200-1000, 200-900, 200-800, 200-700, 200-600, 200-500, 200-400, 200-300, 300-1400, 300-1300, 300-1200, 300-1100, 300-1000, 300-900, 300-800, 300-700, 300-600, 300-500, 300-400, 400-1400, 400-1300, 400-1200, 400-1100, 400-1000, 400-900, 400-800, 400-700, 400-600, 400-500, 500-1400, 500-1300, 500-1200, 500-1100, 500-1000, 500-900, 500-800, 500-700, 500-600, 600-1400, 600-1300, 600-1200, 600-1100, 600-1000, 600-900, 600-800, 600-700, 700-1400, 700-1300, 700-1200, 700-1100, 700-1000, 700-900, 700-800, 800-1400, 800-1300, 800-1200, 800-1100, 800-1000, 800-900, 900-1400, 900-1300, 900-1200, 900-1100, 900-1000, 1000-1400, 1000-1300, 1000-1200, 1000-1100, 1100-1400, 1100-1300, 1100-1200, 1200-1400, or 1200-1300 of a sequence selected from Table A1.

6. The pharmaceutical composition of any one of claims 1-5, wherein (a) is conjugated or fused to an antibody, or an antigen binding fragment thereof.

7. The pharmaceutical composition of claim 6, wherein the antibody, or antigen binding fragment thereof, specifically binds to a tumor-associated antigen (TAA) or tumor-specific antigen (TSA).

8. The pharmaceutical composition of any one of claims 1-7, wherein (b) is selected from a porcine pancreatic elastase (PPE) protein, a human neutrophil elastase (ELANE) protein, a human cathepsin G (CTSG) protein, a human proteinase 3 (PR3) protein, and a granzyme B protein.

9. The pharmaceutical composition of claim 8, wherein:
the PPE protein comprises, consists, or consists essentially of an amino acid sequence that is at least 80, 85, 90, 95, 98, 99, or 100% identical to SEQ ID NO: 5, and which retains the Q211F amino acid substitution;
the PPE protein comprises, consists, or consists essentially of an amino acid sequence that is at least 80, 85, 90, 95, 98, 99, or 100% identical to SEQ ID NO: 6, and which retains the T55A amino acid substitution;
the PPE protein comprises, consists, or consists essentially of an amino acid sequence that is at least 80, 85, 90, 95, 98, 99, or 100% identical to SEQ ID NO: 7, and which retains the Q211F and T55A amino acid substitutions;
the PPE protein comprises, consists, or consists essentially of an amino acid sequence that is at least 80, 85, 90, 95, 98, 99, or 100% identical to SEQ ID NO: 8, and which retains the N241A amino acid substitution;
the PPE protein comprises, consists, or consists essentially of an amino acid sequence that is at least 80, 85, 90, 95, 98, 99, or 100% identical to SEQ ID NO: 9, and which retains the N241Y amino acid substitution;
the PPE protein comprises, consists, or consists essentially of an amino acid sequence that is at least 80, 85, 90, 95, 98, 99, or 100% identical to SEQ ID NO: 10, and which retains the R75A amino acid substitution;
the PPE protein comprises, consists, or consists essentially of an amino acid sequence that is at least 80, 85, 90, 95, 98, 99, or 100% identical to SEQ ID NO: 11, and which retains the R75E amino acid substitution;
the PPE protein comprises, consists, or consists essentially of an amino acid sequence that is at least 80, 85, 90, 95, 98, 99, or 100% identical to SEQ ID NO: 12, and which retains the Q211A amino acid substitution;
the PPE protein comprises, consists, or consists essentially of an amino acid sequence that is at least 80, 85, 90, 95, 98, 99, or 100% identical to SEQ ID NO: 13, and which retains the R237A amino acid substitution;
the PPE protein comprises, consists, or consists essentially of an amino acid sequence that is at least 80, 85, 90, 95, 98, 99, or 100% identical to SEQ ID NO: 14, and which retains the S214A amino acid substitution;
the PPE protein comprises, consists, or consists essentially of an amino acid sequence that is at least 80, 85, 90, 95, 98, 99, or 100% identical to SEQ ID NO: 15, and which retains the D74A amino acid substitution; and
the PPE protein comprises, consists, or consists essentially of an amino acid sequence that is at least 80, 85, 90, 95, 98, 99, or 100% identical to SEQ ID NO: 16.

10. The pharmaceutical composition of claim 8, wherein:
the human ELANE protein comprises, consists, or consists essentially of an amino acid sequence that is at least 80, 85, 90, 95, 98, 99, or 100% identical to SEQ ID NO: 17;
the human CTSG protein comprises, consists, or consists essentially of an amino acid sequence that is at least 80, 85, 90, 95, 98, 99, or 100% identical to SEQ ID NO: 18;
the human PR3 protein comprises, consists, or consists essentially of an amino acid sequence that is at least 80, 85, 90, 95, 98, 99, or 100% identical to SEQ ID NO: 19; or
the human granzyme B protein comprises, consists, or consists essentially of an amino acid sequence that is at least 80, 85, 90, 95, 98, 99, or 100% identical to SEQ ID NO: 20.

11. The pharmaceutical composition of any one of claims 1-10, wherein (a) and (b) are present in the composition at a molar ratio of about 1:3, 1:2.9, 1:2.8, 1:2.7, 1:2.6, 1:2.5, 1:2.4, 1:2.3, 1:2.1, 1:2, 1:1.9, 1:1.8, 1:1.7, 1:1.6, 1:1.5, 1:1.4, 1:1.3, 1:1.2, 1:1.1, or 1:1.

12. The pharmaceutical composition of claim 11, wherein (a) and (b) are present in the composition at a molar ratio of about 1:2.

13. A method of treating, ameliorating the symptoms of, and/or reducing the progression of, a cancer in a subject in need thereof, comprising administering to the subject a pharmaceutical composition of any one of claims 1-12.

14. The method of claim 13, wherein the cancer is a primary cancer or a metastatic cancer, and is selected from one or more of melanoma (optionally metastatic melanoma), breast cancer (optionally triple-negative breast cancer, TNBC), kidney cancer (optionally renal cell carcinoma), pancreatic cancer, bone cancer, prostate cancer, small cell lung cancer, non-small cell lung cancer (NSCLC), mesothelioma, leukemia (optionally lymphocytic leukemia, chronic myelogenous leukemia, acute myeloid leukemia, or relapsed acute myeloid leukemia), multiple myeloma, lymphoma, hepatoma (hepatocellular carcinoma), sarcoma, B-cell malignancy, ovarian cancer, colorectal cancer, glioma, glioblastoma multiforme, meningioma, pituitary adenoma, vestibular schwannoma, primary CNS lymphoma, primitive neuroectodermal tumor (medulloblastoma), bladder cancer, uterine cancer, esophageal cancer, brain cancer, head and neck cancers, cervical cancer, testicular cancer, thyroid cancer, and stomach cancer.

15. The method of claim 13 or 14, wherein administration (optionally intravenous administration) of the pharmaceutical composition does not substantially increase prothrombin time or partial thromboplastin time in the subject.

16. The method of any one of claims 13-15, wherein administration of the pharmaceutical composition increases cancer cell-killing in the subject by about or at least about 2-fold, 5-fold, 10-fold, 50-fold, 100-fold, 500-fold, or 1000-fold or more relative to a control or reference.

17. The method of any one of claims 13-16, comprising administering the pharmaceutical composition to the subject by parenteral administration.

18. The method of claim 17, wherein the parenteral administration is intravenous administration.

19. A method of manufacturing a pharmaceutical composition comprising a protein complex, by combining:
(a) alpha-2-macroglobulin (A2M) proteins; and
(b) serine protease proteins,
into a composition at a molar ratio [(a):(b)] of about 1:3 to about 1:1, thereby manufacturing the pharmaceutical composition comprising the protein complex.

20. The method of claim 19, comprising recombinantly producing (a) prior to combining with (b).

21. The method of claim 19, comprising purifying (a) from plasma of a human subject prior to combining with (b).

22. The method of any one of claims 19-21, comprising recombinantly producing (b) prior to combining with (a).

23. The method of any one of claims 19-22, which comprises combining (a) and (b) at a molar ratio [(a):(b)] of about 1:3, 1:2.9, 1:2.8, 1:2.7, 1:2.6, 1:2.5, 1:2.4, 1:2.3, 1:2.1, 1:2, 1:1.9, 1:1.8, 1:1.7, 1:1.6, 1:1.5, 1:1.4, 1:1.3, 1:1.2, 1:1.1, or 1:1

24. The method of claim 23, which comprises combining (a) and (b) at a molar ratio [(a):(b)] of about 1:2.

25. The method of any one of claims 19-24, wherein the A2M proteins of (a) and the serine protease proteins of (b) are bound together in the protein complex, and optionally wherein the protein complex:
(i) retains CD95 (Fas Receptor) protease cleavage activity and cancer cell-killing activity of (b);
(ii) sterically hinders binding of (b) to fibrinogen and reduces or inhibits fibrinogen cleavage activity of (b); and
(iii) sterically hinders binding of (b) to serine protease inhibitors (including alpha-1 antitrypsin (A1AT)).

26. The method of any one of claims 19-25, wherein (a) comprises, consists, or consists essentially of an amino acid sequence that is at least 80, 85, 90, 95, 98, 99, or 100% identical to a sequence selected from Table A1, or a functional fragment thereof.

27. The method of claim 26, wherein the functional fragment thereof comprises, consists, or consists essentially of about 100, 200, 300, 400, 500, 600, 700, 800, 900, 1000, 1200, 1300, or 1400 contiguous amino acids of a sequence selected from Table A1.

28. The method of claim 27, wherein the functional fragment thereof is composed of approximately residues 1-1400, 1-1300, 1-1200, 1-1100, 1-1000, 1-900, 1-800, 1-700, 1-600, 1-500, 1-400, 1-300, 1-200, 100-1400, 100-1300, 100-1200, 100-1100, 100-1000, 100-900, 100-800, 100-700, 100-600, 100-500, 100-400, 100-300, 100-200, 200-1400, 200-1300, 200-1200, 200-1100, 200-1000, 200-900, 200-800, 200-700, 200-600, 200-500, 200-400, 200-300, 300-1400, 300-1300, 300-1200, 300-1100, 300-1000, 300-900, 300-800, 300-700, 300-600, 300-500, 300-400, 400-1400, 400-1300, 400-1200, 400-1100, 400-1000, 400-900, 400-800, 400-700, 400-600, 400-500, 500-1400, 500-1300, 500-1200, 500-1100, 500-1000, 500-900, 500-800, 500-700, 500-600, 600-1400, 600-1300, 600-1200, 600-1100, 600-1000, 600-900, 600-800, 600-700, 700-1400, 700-1300, 700-1200, 700-1100, 700-1000, 700-900, 700-800, 800-1400, 800-1300, 800-1200, 800-1100, 800-1000, 800-900, 900-1400, 900-1300, 900-1200, 900-1100, 900-1000, 1000-1400, 1000-1300, 1000-1200, 1000-1100, 1100-1400, 1100-1300, 1100-1200, 1200-1400, or 1200-1300 of a sequence selected from Table A1.

29. The method of any one of claims 19-28, wherein (a) is conjugated or fused to an antibody, or an antigen binding fragment thereof.

30. The method of claim 29, wherein the antibody, or antigen binding fragment thereof, specifically binds to a tumor-associated antigen (TAA) or a tumor-specific antigen (TSA).

31. The method of any one of claims 19-30, wherein (b) is selected from a porcine pancreatic elastase (PPE) protein, a human neutrophil elastase (ELANE) protein, a human cathepsin G (CTSG) protein, a human proteinase 3 (PR3) protein, and a human granzyme B protein.

32. The method of claim 31, wherein:
the PPE protein comprises, consists, or consists essentially of an amino acid sequence that is at least 80, 85, 90, 95, 98, 99, or 100% identical to SEQ ID NO: 5, and which retains the Q211F amino acid substitution;
the PPE protein comprises, consists, or consists essentially of an amino acid sequence that is at least 80, 85, 90, 95, 98, 99, or 100% identical to SEQ ID NO: 6, and which retains the T55A amino acid substitution;
the PPE protein comprises, consists, or consists essentially of an amino acid sequence that is at least 80, 85, 90, 95, 98, 99, or 100% identical to SEQ ID NO: 7, and which retains the Q211F and T55A amino acid substitutions;
the PPE protein comprises, consists, or consists essentially of an amino acid sequence that is at least 80, 85, 90, 95, 98, 99, or 100% identical to SEQ ID NO: 8, and which retains the N241A amino acid substitution;
the PPE protein comprises, consists, or consists essentially of an amino acid sequence that is at least 80, 85, 90, 95, 98, 99, or 100% identical to SEQ ID NO: 9, and which retains the N241Y amino acid substitution;
the PPE protein comprises, consists, or consists essentially of an amino acid sequence that is at least 80, 85, 90, 95, 98, 99, or 100% identical to SEQ ID NO: 10, and which retains the R75A amino acid substitution;
the PPE protein comprises, consists, or consists essentially of an amino acid sequence that is at least 80, 85, 90, 95, 98, 99, or 100% identical to SEQ ID NO: 11, and which retains the R75E amino acid substitution;
the PPE protein comprises, consists, or consists essentially of an amino acid sequence that is at least 80, 85, 90, 95, 98, 99, or 100% identical to SEQ ID NO: 12, and which retains the Q211A amino acid substitution;
the PPE protein comprises, consists, or consists essentially of an amino acid sequence that is at least 80, 85, 90, 95, 98, 99, or 100% identical to SEQ ID NO: 13, and which retains the R237A amino acid substitution;
the PPE protein comprises, consists, or consists essentially of an amino acid sequence that is at least 80, 85, 90, 95, 98, 99, or 100% identical to SEQ ID NO: 14, and which retains the S214A amino acid substitution;
the PPE protein comprises, consists, or consists essentially of an amino acid sequence that is at least 80, 85, 90, 95, 98, 99, or 100% identical to SEQ ID NO: 15, and which retains the D74A amino acid substitution; and
the PPE protein comprises, consists, or consists essentially of an amino acid sequence that is at least 80, 85, 90, 95, 98, 99, or 100% identical to SEQ ID NO: 16.

33. The method of claim 31, wherein:
the human ELANE protein comprises, consists, or consists essentially of an amino acid sequence that is at least 80, 85, 90, 95, 98, 99, or 100% identical to SEQ ID NO: 17;
the human CTSG protein comprises, consists, or consists essentially of an amino acid sequence that is at least 80, 85, 90, 95, 98, 99, or 100% identical to SEQ ID NO: 18;
the human PR3 protein comprises, consists, or consists essentially of an amino acid sequence that is at least 80, 85, 90, 95, 98, 99, or 100% identical to SEQ ID NO: 19; or
the human granzyme B protein comprises, consists, or consists essentially of an amino acid sequence that is at least 80, 85, 90, 95, 98, 99, or 100% identical to SEQ ID NO: 20.

34. The method of any one of claims 19-33, further comprising the step of testing the pharmaceutical composition in one or more activity assays selected from one or more of a CD95 cleavage assay (optionally in the presence of a serine protease inhibitor such as ALAT), a fibrinogen cleavage assay, and a cancer cell-killing assay.

35. The method of claim 32, wherein the pharmaceutical composition cleaves CD95 (optionally in the presence of the serine protease inhibitor such as ALAT), does not substantially cleave fibrinogen, and/or has cancer cell-killing activity.


SEQUENCE LISTING (23 sequences)

SEQ ID NO: 1 - protein, 266 aa, Homo sapiens
MLRLLVVASLVLYGHSTQDFPETNARVVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 2 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 3 - protein, 255 aa, Homo sapiens
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 4 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 5 - protein, 240 aa, synthetic construct
VVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCFGDSGGPLHCLVNGQYAVHGVTSFVSRLGCNVTRKPTVFTRVSAYISWINNVIASN

SEQ ID NO: 6 - protein, 240 aa, synthetic construct
VVGGTEAQRNSWPSQISLQYRSGSSWAHACGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 7 - protein, 240 aa, synthetic construct
VVGGTEAQRNSWPSQISLQYRSGSSWAHACGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCFGDSGGPLHCLVNGQYAVHGVTSFVSRLGCNVTRKPTVFTRVSAYISWINNVIASN

SEQ ID NO: 8 - protein, 240 aa, synthetic construct
VVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCAVTRKPTVFTRVSAYISWINNVIASN

SEQ ID NO: 9 - protein, 240 aa, synthetic construct
VVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCYVTRKPTVFTRVSAYISWINNVIASN

SEQ ID NO: 10 - protein, 240 aa, synthetic construct
VVGGTEAQRNSWPSQISLQYRSGSSWAHTCGGTLIRQNWVMTAAHCVDAELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 11 - protein, 240 aa, synthetic construct
VVGGTEAQRNSWPSQISLQYRSGSSWAHTCGGTLIRQNWVMTAAHCVDEELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 12 - protein, 240 aa, synthetic construct
VVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCAGDSGGPLHCLVNGQYAVHGVTSFVSRLGCNVTRKPTVFTRVSAYISWINNVIASN

SEQ ID NO: 13 - protein, 240 aa, synthetic construct
VVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSALGCNVTRKPTVFTRVSAYISWINNVIASN

SEQ ID NO: 14 - protein, 240 aa, synthetic construct
VVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDAGGPLHCLVNGQYAVHGVTSFVSRLGCNVTRKPTVFTRVSAYISWINNVIASN

SEQ ID NO: 15 - protein, 240 aa, synthetic construct
VVGGTEAQRNSWPSQISLQYRSGSSWAHTCGGTLIRQNWVMTAAHCVARELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 16 - protein, 240 aa, Sus scrofa
VVGGTEAQRNSWPSQISLQYRSGSSWAHTCGGTLIRQNWVMTAAHCVDRELTFRVVVGEHNLNQNDGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGDGVRSGCQGDSGGPLHCLVNGQYAVHGVTSFVSRLGCNVTRKPTVFTRVSAYISWINNVIASN

SEQ ID NO: 17 - protein, 238 aa, Homo sapiens
IVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 18 - protein, 235 aa, Homo sapiens
IIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 19 - protein, 229 aa, Homo sapiens
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 20 - protein, 227 aa, Homo sapiens
IIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 21 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 22 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 23 - protein, 1451 aa, Homo sapiens
SVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA